(19) United States
(12) Patent Application Publication
(10) Pub. No.: US 2012/0045487 A1
(43) Pub. Date: Feb. 23, 2012

(54) MULTIPHASIC MICROFIBERS FOR SPATIALLY GUIDED CELL GROWTH

(75) Inventors: Joerg Lahann, Ann Arbor, MI (US); Srijanani Bhaskar, Ann Arbor, MI (US); Suparna Mandal, Ann Arbor, MI (US)

(73) Assignee: The Regents of the University of Michigan, Ann Arbor, MI (US)

(21) Appl. No.: 13/266,377

(22) PCT Filed: Apr. 29, 2010
(86) PCT No.: PCT/US2010/032971

§ 371 Date: Oct. 26, 2011

Related U.S. Application Data
(60) Provisional application No. 61/173,864, filed on Apr. 29, 2009.

(51) Int. Cl. A61K 47/34; A61K 45/00; A61K 38/21; A61K 38/20; C12N 5/071; A61P 37/02; A61P 29/00; A61P 35/00; A61P 31/00; A61P 31/10; A61P 31/12; C08G 63/91; A61K 38/02; A61K 31/7088; A61K 31/715; A61K 31/70; A61K 38/18; A61K 38/30; A61K 38/19; A61K 9/00; A61P 7/02; A61P 39/06; A61P 23/00; A61P 9/04; A61P 5/28; A61P 5/36; B29B 9/06; A61K 39/395; B82Y 5/00

(52) U.S. Cl. 424/400; 424/130.1; 424/94.1; 424/85.4; 424/85.2; 435/396; 525/415; 514/772.1; 514/19.1; 514/1.1; 514/44 R; 514/54; 514/23; 514/7.7; 514/8.1; 514/8.9; 514/9.1; 514/9.6; 514/7.6; 514/8.4; 514/9.2; 514/8.5; 514/8.2; 424/85.1; 264/10; 977/774; 977/915; 977/700

(57) ABSTRACT

A multiphasic microfiber for a three-dimensional tissue scaffold and/or cellular support is provided in one aspect that includes at least one biocompatible material. The multiphasic microfiber optionally has a first phase and at least one distinct additional phase and is formed by electrohydrodynamic jetting. Further, such microfibers optionally have one or more biofunctional agents, which may be surface-bound moieties provided in spatial patterns. Multiphasic microfibers formed in accordance with the disclosure may form, in some cases, three-dimensional fiber scaffolds with precisely engineered, micrometer-scaled patterns for cellular contact guidance, which may thus support and/or promote cellular growth, proliferation, differentiation, repair, and/or regeneration for tissue and bioengineering applications.

### CROSS-REFERENCE TO RELATED APPLICATIONS

This application claims the benefit of U.S. Provisional Application No. 61/173,864, filed on Apr. 29, 2009. This application is also a continuation-in-part of pending U.S. patent application Ser. No. 12/257,945 filed on Oct. 24, 2008, which claims the benefit of U.S. Provisional Application No. 60/982,389 filed on Oct. 24, 2007, and which is a continuation-in-part of pending U.S. patent application Ser. No. 11/272,194 filed on Nov. 10, 2005, which claims the benefit of U.S. Provisional Application No. 60/626,792 filed on Nov. 10, 2004, and the benefit of U.S. Provisional Application No. 60/651,288, filed Feb. 9, 2005.

The entire disclosures of each of the above applications are incorporated herein by reference.

### FIELD

The present disclosure relates to the fabrication of microfibers and, more particularly, to methods of fabricating multiphasic microfiber scaffolds for promoting spatially guided cell growth and proliferation.

### BACKGROUND

This section provides background information generally related to the present disclosure which is not necessarily prior art.

The nano- and microstructure of the cellular microenvironment is a decisive factor related to many biological phenomena important for regenerative medicine, such as cell morphology, adhesion, motility, or apoptosis. Mimicking surfaces with natural, spatially continuous gradients is therefore important to a range of biological applications, including neuronal growth and differentiation, the design of cell migration, inflammation assays, microfluidics, and discovery-driven biomaterials research. In spite of the importance of surface gradients and multidimensional spatial patterning for biological applications, their realization, especially with biomedically relevant polymers, has been challenging. Because the local microstructure plays a pivotal role for many biological functions, a wide range of methods have been developed to design precisely engineered substrates for both fundamental biological studies and biotechnological applications. However, these techniques have been by-and-large limited to flat surfaces.

Towards this end, some natural and synthetic functional polymers can produce micro- and nanofibers, which can provide three-dimensional cellular support structures. However, such functional polymers provide little control over local spatial geometry, which is believed to be as important as the material composition of the tissue scaffolding or cellular support substrates. Successful methods for micropatterning of conventional three-dimensional fiber scaffolds with biomolecules, such as cell adhesion peptides, are not presently available. Thus, it would be desirable to have a scaffold structure for cell proliferation that includes one or more biocompatible micro or nanofibers having highly controlled spatial geometry and alignment, multiple phases or compartments having one or more biomolecules or biologically active materials that enhance cell proliferation and/or are biofunctional in the surrounding environment.

### SUMMARY

This section provides a general summary of the disclosure, and is not a comprehensive disclosure of its full scope or all of its features.

In various aspects, the present disclosure provides a multiphasic microfiber defining a longitudinal major axis and comprising at least one biocompatible material. The multiphasic microfiber also comprises a first phase and at least one additional phase distinct from the first phase. Further, at least a portion of the first phase and at least one additional phase have exposed surfaces to an external surrounding environment. In certain variations, the multiphasic microfiber further comprises at least one biofunctional agent, as well. Such multiphasic microfibers support and/or promote cell growth, cell proliferation, cell differentiation, cell repair, and/or cell regeneration.

In other aspects, the disclosure provides a three-dimensional cellular scaffold structure comprising at least two multiphasic microfibers respectively defining an evident longitudinal major axis and respectively comprising a first phase and at least one additional phase distinct from the first phase. At least a portion of the first phase and the at least one additional phase of each respective multiphasic microfiber has an exposed surface to an external surrounding environment and comprises a biocompatible material. In this manner, the cellular scaffold structure supports and/or promotes cell growth, cell proliferation, cell differentiation, cell repair, and/or cell regeneration in three-dimensions.

In yet other aspects, the present disclosure provides methods of making a multiphasic microfiber for a tissue scaffold and/or cellular support structure. In certain aspects, the method comprises forming a plurality of multiphasic microfibers by jetting two or more liquid streams together and passing them through an electric field generated by electrodes sufficient to form a cone jet that forms the plurality of microfibers. Each of the microfibers respectively has a first phase and at least one additional phase distinct from the first phase, which form exposed surfaces that are exposed to an external surrounding environment. Each multiphasic microfiber respectively comprises a biocompatible material for supporting and/or promoting cell growth, cell proliferation, cell differentiation, cell repair, and/or cell regeneration.

Further areas of applicability will become apparent from the description provided herein. The description and specific examples in this summary are intended for purposes of illustration only and are not intended to limit the scope of the present disclosure.

### DRAWINGS

The drawings described herein are for illustration purposes only and are not intended to limit the scope of the present disclosure in any way.

FIG. 1 is an exemplary apparatus according to the present disclosure for electrically jetting fluid in a side-by-side configuration to form multiphasic nano-component fibers;

FIG. 2 shows the relationship between flow rate and concentration on nano-component shapes during electrified jetting of a poly(lactide-co-glycolide) polymer (PLGA) in accordance with the principles of the present disclosure;

FIG. 3 is a diagram showing the relationship of molecular weight and concentration to morphology;

FIGS. 4A and 4B are Confocal Laser Scanning Micrographs (CLSM) according to various principles of the present teachings depicting different shapes of fibers and elongated rod shaped particles;

FIGS. 5A and 5B are scanning electron microscopy micrographs (SEM). FIG. 5A is an SEM (having a scale of about 0.05 mm) of a highly aligned tissue scaffold formed by multiphasic microfibers formed according to various principles of the present teachings. FIG. 5B is an SEM (having a scale of about 0.2 mm) of a highly aligned biphasic tissue scaffold formed by multiphasic microfibers principles of the present teachings;

FIGS. 6A through 6M show various multiphasic nano-components formed in accordance with the methods of the present disclosure with biodegradable PLGA polymers, including SEM and CLSM images of various aligned multiphasic microfibers;

FIGS. 7A-7I are schematic depictions of cross-sectional morphologies of various multiphasic microfibers (having from three to seven phases) with respective insets showing jetting apparatus stream configurations;

FIGS. 8A-8C show comparative images and schematics of multiphasic microfibers by selective modification with a biofunctional surface moiety (a laminin-derived IKVAV pentapeptide sequence of Ile-Lys-Val-Ala-Val for cell adhesion) with CLSM images shown along with insets representing the corresponding phase contrast images;

FIGS. 9A-9F show optical and CLSM micrographs of NIH 3T3 fibroblasts cultured on biphasic PLGA microfibers selectively immobilized and modified with IKVAV-peptide in accordance with the present teachings;

FIG. 10 shows exemplary schematics of two multiphasic microfibers substantially aligned with one another along a major axis “a”; and

FIG. 11 shows an exemplary schematic of alignment of various multiphasic microfibers along a substrate surface deposited in accordance with the present teachings;

FIGS. 12A-12L show top view CLSM micrographs (at a scale of 0.2 mm) of tricompartmental microfiber scaffolds created by side-by-side co-jetting of three different PLGA solutions, where individual blue (B), green (G), and red (R) micrographs representing fluorescence from poly[(mphenylenevinylene)-alt-(2,5-dibutoxy-p-phenylenevinylene)] (MEHPPV), Poly[tris(2,5-bis(hexyloxy)-1,4-phenylenevinylene)-alt-(1,3-phenylenevinylene)] (PTDPV), and region regular poly(3-hexyl-thiophene-2,5-diyl) polymer (ADS306PT) dyes (each shown independently, where blue is shown in FIGS. 12A, 12E, and 12I, green in FIGS. 12B, 12F, and 12J, and red in FIGS. 12C, 12G, and 12K, followed by an overlay of the three individual fluorescence images (from left to right in composites FIGS. 12D, 12H, and 12L; and

FIGS. 13A-13C show multiphasic microfibers formed in accordance with the present teachings, where in FIG. 13A, cell attachment (NIH3T3 fibroblasts) occurs on surfaces of a multiphasic microfiber comprising two PLGA phases, as where FIGS. 13B and 13C show a multiphasic microfiber comprising a first PLGA phase and a second polyethylene glycol (PEG)-PLGA phase, where selective adhesion of the cells occurs on only the PLGA phase.

Corresponding reference numerals indicate corresponding parts throughout the several views of the drawings.

### DETAILED DESCRIPTION

When an element or layer is referred to as being “on,” “engaged to,” “connected to” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, phases, regions, layers and/or sections, these elements, components, phases, regions, layers and/or sections should not be limited by these terms. These terms may be only used to distinguish one element, component, phase, region, layer or section from another region, layer or section. Terms such as “first,” “second,” and other numerical terms when used herein do not imply a sequence or order unless clearly indicated by the context. Thus, a first element, component, phase, region, layer or section discussed below could be termed a second element, component, phase, region, layer or section without departing from the teachings of the example embodiments.

Spatially relative terms, such as “inner,” “outer,” “beneath,” “below,” “lower,” “above,” “upper” and the like, may be used herein for ease of description to describe one element or feature's relationship to another element(s) or feature(s) as illustrated in the Figures. Spatially relative terms may be intended to encompass different orientations of the components as formed or in use in addition to the orientation depicted in the Figures. For example, if the fibers or scaffolds of the Figures are turned over, elements described as “below” or “beneath” other elements or features would then be oriented “above” the other elements or features. Thus, the example term “below” can encompass both an orientation of above and below. The scaffolds or phases may be otherwise oriented (rotated 90 degrees or at other orientations) and the spatially relative descriptors used herein interpreted accordingly.

Exemplary embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

In various aspects, the present teachings provide a multiphasic biocompatible microfiber for promoting cell growth, proliferation, differentiation, repair, and/or regeneration. By “fiber” it is meant that the component defines an evident longitudinal axis and thus has a so-called “axial geometry.” Fibers having such an evident longitudinal axis include an elongated axial dimension, which is longer than the other dimensions (e.g., diameter or width) of the fiber. In certain aspects, such elongated fiber components having an axial geometry have an aspect ratio (AR) defined as a length of the longest axis divided by diameter of the component, which is preferably at least about 100 and in certain aspects greater than about 1,000. In yet other aspects, such fibers may have an aspect ratio of 10,000 or more.

A “microfiber” as used herein encompasses “nanofibers,” as discussed below. A microfiber component has an evident longitudinal axis or axial geometry, as defined above, and further has at least one spatial dimension that is less than about 1,000 μm (i.e., 1 mm), optionally less than or equal to about 100 μm (i.e., 100,000 nm). The term “micro-sized” or “micrometer-sized” as used herein is generally understood by those of skill in the art to mean less than about 500 μm (i.e., 0.5 mm). As used herein, a microfiber component has at least one spatial dimension that is less than about 100 μm (i.e., 100,000 nm), optionally less than about 50 μm (i.e., 50,000 nm), optionally less than about 10 μm (i.e., 10,000 nm), and in certain aspects less than or equal to about 5 μm (i.e., 5,000 nm). In certain aspects, a microfiber component has at least one spatial dimension that is less than or equal to about 1,000 μm, optionally less than or equal to about 100 μm, optionally less than or equal to about 50 μm, and in certain embodiments, less than or equal to 10 μm.

Fibers that are “nano-sized” or “nanometer-sized” as used herein are generally understood by those of skill in the art to have at least one spatial dimension that is less than about 50 μm (i.e., 50,000 nm), optionally less than about 10 μm (i.e., 10,000 nm), optionally less than about 2 μm (i.e., less than about 2,000 nm), optionally less than or equal to about 1 μm, optionally less than about 0.5 μm (i.e., 500 nm), and in certain aspects, less than about 200 nm. Accordingly, a nanofiber component has at least one spatial dimension that is greater than about 1 nm and less than about 50,000 nm (50 μm). In certain variations, a nanofiber may have at least one spatial dimension of about 5 nm to about 5,000 nm. In certain variations, at least one spatial dimension of the nanofiber component is about 20 nm to about 2,000 nm. In still other variations, nanofiber components have at least one spatial dimension of about 50 nm to about 500 nm. Such nanofiber components are intended to encompass components having a micro-scale, so long as at least one dimension of the fiber is less than about 50 μm. It should be noted that so long as at least one dimension of the nanofiber falls within the above-described nano-sized scale (for example, diameter), one or more other axes may well exceed the nano-size (for example, length and/or width). As used herein, unless otherwise indicated, the terms micro-component and nano-component, or microfiber and nanofiber, are used interchangeably.

In various aspects, the microfibers of the present disclosure comprise a first phase and at least one additional phase distinct from the first phase. Furthermore, at least one of the first phase and one or more additional phases comprises a biocompatible material such as a polymer and/or an agent for interacting with cells to promote cell growth, proliferation, differentiation, repair, and/or regeneration.

By the term “phase” it is meant that a portion of a microfiber component is chemically and/or physically distinct from another portion of the microfiber component. The multiphasic microfibers according to the present teachings include a first phase and at least one phase that is distinct from the first phase. In certain aspects, the multiphasic microfibers of the present disclosure include multiple distinct phases, for example three or more distinct phases. It should be noted that distinct phases in the microfibers may include phases having the same composition that physically occupy different portions of the microfiber, in other words that certain phases, although physically distinct from one another, may chemically be the same and repeated in the fiber. In certain aspects, the microfiber may comprise multiple phases, ranging from two to hundreds of distinct phases.

In some aspects, each respective phase occupies a spatially discrete region or compartment of the microfiber, such as an elongated spatial region along the elongate longitudinal major axis forming an elongate component. In certain aspects, at least a portion a first phase and at least a portion of another phase distinct from the first phase are exposed to an external environment. In yet other aspects, each respective phase of the multiphasic component is exposed to an external environment, thus providing exposure of the respective phase surfaces of the multiphasic fiber to an external environment. In various aspects, the exposure of respective phase surfaces provides enhanced environmental interface and optimum diffusion or material transfer, resulting in increased bioavailability to target regions.

In certain aspects, each respective phase that is present within the microfiber is aligned or substantially aligned along an evident longitudinal major axis. In certain variations, microfiber components comprise materials in a solid phase or a semi-solid phase, although liquid phases are contemplated in certain variations. In certain alternate aspects, the microfiber may have a first core phase surrounded by at least one additional phase (e.g., one or more shell or encasing phases). In such embodiments, the microfiber may have a solid shell phase and a liquid or semi-liquid core phase, for example.

Conventional tissue engineering scaffolds have been composed of porous polymer fiber networks that act as substrates for cell attachment. However, more complex architectures that mimic conventional tissue structures have been more difficult to produce. The ultimate goal of a scaffold is to replace or restore physiological functions which have been lost in diseased or damaged organs. Mimicking the micro-architecture of tissues and the microenvironment around cells within the body has been shown to be an important criterion for increasing the functionality of a tissue engineering construct, because directionality is important for the presentation of vital biochemical and physical cues that determine cellular fate through processes such as proliferation, differentiation, migration, and apoptosis. This is particularly true in the case of neurons. This control over microenvironment has previously been provided through two dimensional patterned substrates.

The present inventive multiphasic microfibers provide new techniques and technology that combines patterning and distinct internal architectures that can be used to create three-dimensional substrates or scaffolds for spatially directed cell growth. Scaffold structures can be built with such multiphasic microfibers, which are then optionally patterned with cell adhesion ligands over controlled areas. This control over distribution of cell biofunctional moieties, such as adhesion ligands, or other biofunctional agents as well as internal architecture of the multiphasic microfiber introduces new design parameters for scaffold design, and facilitates a better understanding of dependence of cell fate on directional cues.

The multiphasic microfibers and multiphasic fiber scaffolds created from a plurality of the multiphasic fibers made in accordance with the principles of the present disclosure provide enhanced three-dimensional spatial control, including the ability to provide enhanced “surface patterning,” enabling highly selective cell guidance at superior spatial pattern resolutions that have only previously been observed for flat substrates.

The spatial pattern resolution can be defined in terms of the fiber diameter (y) and number of phases or compartments (x). For a fiber with x phases and a diameter of y, the spatial resolution is y/x. For example, where the microfiber has a diameter of 20 micrometers and 2 phases/compartments, the spatial resolution is 10 μm. Expressed in another way, the resolution represents the area on which a moiety (such as a biofunctional moiety) is selectively immobilized. In accordance with certain aspects of the present disclosure, the microfibers formed have a spatial pattern resolution of less than or equal to about 100 μm, optionally less than or equal to about 50 μm, optionally less than or equal to about 30 μm, optionally less than or equal to about 25 μm, optionally less than or equal to about 20 μm, optionally less than or equal to about 15 μm, optionally less than or equal to about 10 μm. In certain variations, a microfiber formed in accordance with the present disclosure has a spatial resolution of greater than or equal to about 0.1 μm (100 nm) to less than or equal to about 100 μm, optionally greater than or equal to about 2 μm to less than or equal to about 30 μm, optionally greater than or equal to about 5 μm to less than or equal to about 20 μm, and in certain aspects, optionally greater than or equal to about 8 μm to less than or equal to about 10 μm.

The present teachings pertain in one aspect to methods of forming multiphasic microfibers that have a high degree of control or selectivity with respect to the compositions, size, spatial position, morphology, and alignment of respective phases (a first phase and/or at least one additional phase) when forming a plurality of microfiber components via electrospraying techniques described herein. Such methods include jetting two or more liquid streams together and passing them through an electric field generated by electrodes sufficient to form a cone jet that forms the plurality of microfiber components. In this regard, such multiphasic microfibers can be used as a substrate or scaffold compatible with biological systems to promote spatially guided cell growth.

As discussed above, each microfiber respectively has a first phase and at least one additional phase distinct from the first phase. In various aspects, the materials selected for the microfibers are preferably biocompatible, in other words, substantially non-toxic to cells and tissue of living organisms, as will be described in more detail below. As will be described in greater detail below, certain biofunctional agents may be included in the microfiber components that are selected to have toxicity to certain target cells (e.g., anti-proliferative agents, like chemotherapeutic agents) or organisms (e.g., antimicrobial agents), or may be selected for having certain specified benefits to cells, tissue, or an organism that outweigh potential detrimental impact in a conventional risk-benefit assessment.

In certain aspects, the present disclosure provides methods to form such multiphasic microfibers by controlling one or more of: concentration of the polymer in the liquid streams, flow rate of the liquid streams, humidity, temperature, electrode design, and configuration of electrodes during the jetting process, to provide a high selectivity of particles formed that have substantially the same shape, size, and orientation of a first phase and/or at least one additional phase. For example, in certain aspects, concentration of polymer and flow rates of the liquid streams are two significant variables controlled while forming a plurality of microfiber components, which have substantially the same shape, size, or phase orientation. In other aspects, the electrode geometry and configuration during the electrospraying process is employed to control microfiber size, shape, selectivity, and distribution.

Moreover, orientation, size, and arrangement of the fiber phases are controlled in accordance with the present teachings in a highly predictable fashion. Such microfibers can be used to form substrates that are compatible with cells to form three-dimensional scaffolds or cellular support structures. Micro-structured fiber scaffolds have utility for a range of biotechnological applications, including tissue engineering and medical implants or cell-based assays, for example. Thus, in certain aspects, a three-dimensional scaffold structure for promoting cell growth, cell repair, and/or cell regeneration is contemplated by the present disclosure. In certain aspects, the microfibers or nanofibers formed by the teachings of the present disclosure define an evident longitudinal major axis and comprise a first phase and at least one additional phase distinct from the first phase, as described above. Further, at least one of the first phase and the at least one additional phase (and optionally both the first and at least one additional) comprises a biocompatible polymer. In certain aspects, the first phase and/or the at least one additional phase(s) optionally comprises a material or agent that interacts with cells to promote cell growth, proliferation, differentiation, repair, and/or regeneration. For example, in certain embodiments, the three-dimensional scaffold structure promotes directed cellular proliferation in three-dimensions.

In accordance with the present teachings, the microfibers are formed via electrified jetting, a process that develops liquid jets having a nano- and micro-sized particle diameter using electrohydrodynamic forces. When a droplet of electrically conductive solution is exposed to an electric potential (for example, on the order of a few kilovolts), the balance of forces between the electric field and surface tension causes the meniscus of the droplet to distort into a conical shape, called the Taylor cone. Above a specific critical potential value, a highly charged liquid jet is ejected from the apex of the cone. As provided by the present disclosure, a large number of solution and process variables can be manipulated to consistently yield a variety of conformations, phase orientation, and sizes of fibers formed in this manner.

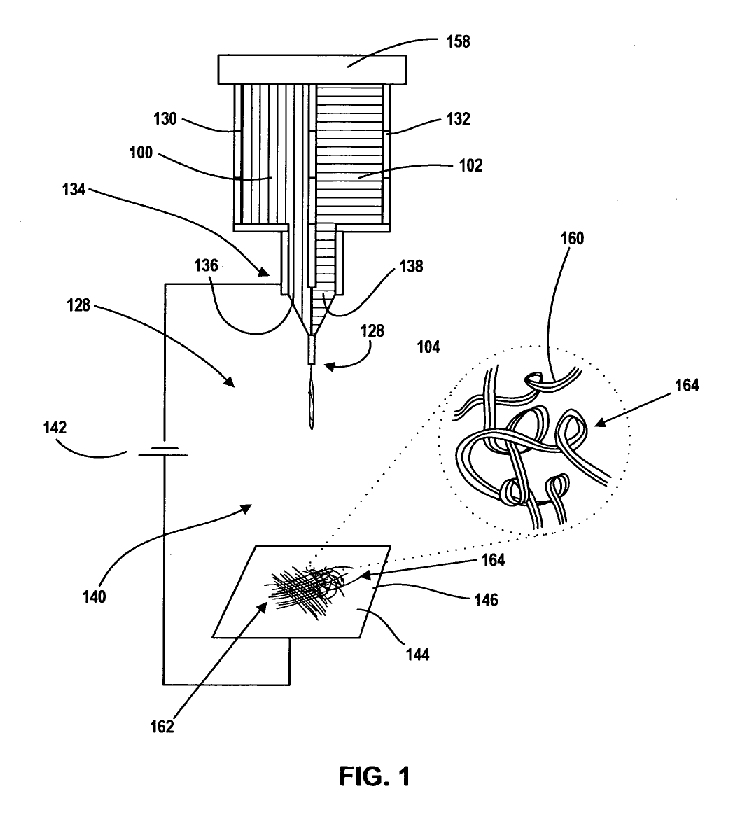

With reference to FIG. 1, a side-by-side electrojetting apparatus illustrates a variation of the method of the disclosure employing polymer solutions or melts as jetting liquid streams. FIG. 1 illustrates a variation of an electrojetting apparatus where two jetting liquids are combined to form microfibers when polymer solutions or melts are used as jetting liquids, fibers 160 are obtained.

In FIG. 1, a “side-by-side” configuration of Fluids A and B 100, 102 are combined to form a pendant droplet 104 of conducting liquid. The two Fluids A and B in FIG. 1 are merely exemplary and non-limiting, as multiple fluids can be jetted to form a plurality of phases depending on the fibers desired, as described further below. The drop 104 is exposed to an electric potential 142 of a few kilovolts, where the force balance between electric field and surface tension causes the meniscus of the pendent droplet 104 to develop a conical shape, the so-called Taylor cone (not shown). Above a critical point, a highly charged liquid jet is ejected from an apex of the cone.

As schematically presented in FIG. 1, the biphasic jet that is ejected by the stable biphasic cone is continuous (i.e., not fragmented) and can solidify into biphasic microfibers. The two phases, i.e., the two jetting liquid streams (or solutions), are optionally compatible with each other (e.g., miscible or soluble) or in certain alternate variations are incompatible. Where the two polymer solutions are compatible with each other, a stable cone-jet forms a stable interface between the two phases. In such situations, it is believed that the process is kinetically controlled (rather than thermodynamically controlled), resulting in one phase being trapped in each side before they mix with the other phase.

Each side of the composite stream 128, channels 130, 132 is configured adjacent to each other (i.e., side by side) in nozzle 134. As noted above, the setup of the electrified jetting apparatus is exemplary and not limited in number of channels or configuration of the respective channels. A syringe pump (not shown) is used to drive the liquids in nozzle 134. In some variations, channels 130, 132 are capillaries. Channels 130, 132 feed two different jetting liquid streams 136, 138 into region 140 having an electric field generated by power supply 142. Channels 130, 132 are of sufficient dimensions to allow contact of liquids streams 100, 102 to drop 104, which forms composite stream 144. In one variation, this electric field is generated by the potential difference between nozzle 134 and receiving substrate plate 146. Typically, an electric field is formed by applying a potential difference between at least two electrodes from about 0.1 kV to about 25 kV. Various configurations of plates and geometries (electrodes) may be used to generate the electric field as known to those of skill in the art and are contemplated by the present disclosure.

Since the electrified jetting methods are related to electrohydrodynamic processes, the properties of the jetting liquid and operating parameters are interrelated. Moreover, when the jetting liquids are not one-component systems (i.e., mixtures of two or more compounds), the jetting liquid is a solution having properties governed by several parameters of the solvent and solutes. It should be appreciated that liquid properties, solution parameters, and operating parameters are related, as recognized by those of skill in the art. Relevant material properties that affect the fibers formed include viscosity, surface tension, volatility, thermal and electrical conductivity, dielectric permittivity, and density. Relevant solution properties include polymer concentrations, molecular weight of polymer, solvent mixtures, surfactant(s), doping agent(s), and cross-linking agent(s). Relevant operating parameters include flow rate of the liquid streams, electric potential, temperature, humidity, and ambient pressure. With regard to the operating parameters, the average size and size distributions of the droplets in electrospraying with cone-jet mode is generally dependent on the flow rate (pumping rate of the jetting liquids).

At a fixed flow rate, one or several relatively monodisperse classes of nano-component diameters are formed. At minimum flow rates, the modality of the distributions and diameter of the droplet itself also show their minima. When the flow rate is changed, the electric field can be adjusted by changing either distance or electric potential between the electrodes in order to sustain a stable cone-jet mode. Higher flow rates may be accompanied by a higher electrical field applied for mass balance of jetting liquids.

In certain aspects, the process dependent variables which are used to control particle shape to arrive at a predetermined multiphasic microfiber shape, include, but are not limited to, concentration of polymers in and conductivity of the respective jetting solutions, as well as flow rates of the jetting streams. The concentration of a polymer (along with other components) in a solution/jetting stream influences the viscosity, as does the molecular weight of the polymer (and other components, where present). Solvents or vehicles used in the jetting solution impact the dielectric constant of a respective jetting stream, viscosity, and vapor pressure. The flow rate of the jetting liquid stream relates to vapor pressure and stability of the jet formed. In certain aspects, the distance between a collector and a needle tip impacts the strength of the electric field applied, which in turn can impact the stability of the cone, as well as the cone shape itself and thus voltage, formed during jetting. Generally, so long as a stable cone jet is formed via correct distance between the electrode and the nozzle/needle tip, this variable does not have a significant impact on nano-component particle shape. Temperature, pressure, and humidity likewise impact the behavior of the jetting fluids and shapes formed, impacting solvent volatilization and applied voltage, for example. In accordance with various aspects of the present teachings, the process variables are controlled in a manner that forms a fibrous shape having consistent phase alignment, length, and/or diameter.

The set of capillaries is arranged in a side-by-side configuration. Under these conditions, a well-defined interface can be formed within the pendant droplet. Upon application of a sufficiently high threshold voltage, accumulation of surface charges results in the formation of a liquid cone. The liquid cone acts as the origin of a polymer jet that retains the multiphasic geometry of the initial droplet through jet elongation, solvent evaporation, and polymer solidification. As a result of the formation of a stable jet having laminar flow, multiphasic fibers can be deposited onto a counter electrode in a highly aligned fashion (see region 162 of FIG. 1). Alternately, the fibers can be provided in a random pattern, by orienting the substrate plate 146 in different directions, for example. See region 164 of FIG. 1. In these biphasic microfibers, individual phases or compartments can differ with respect to their chemical compositions, which can be controlled by controlling the composition of the initial jetting solutions. Thus, individual phases may be comprised of a variety of different additives, such as functional polymers, dyes, biomolecules, and/or active agents.

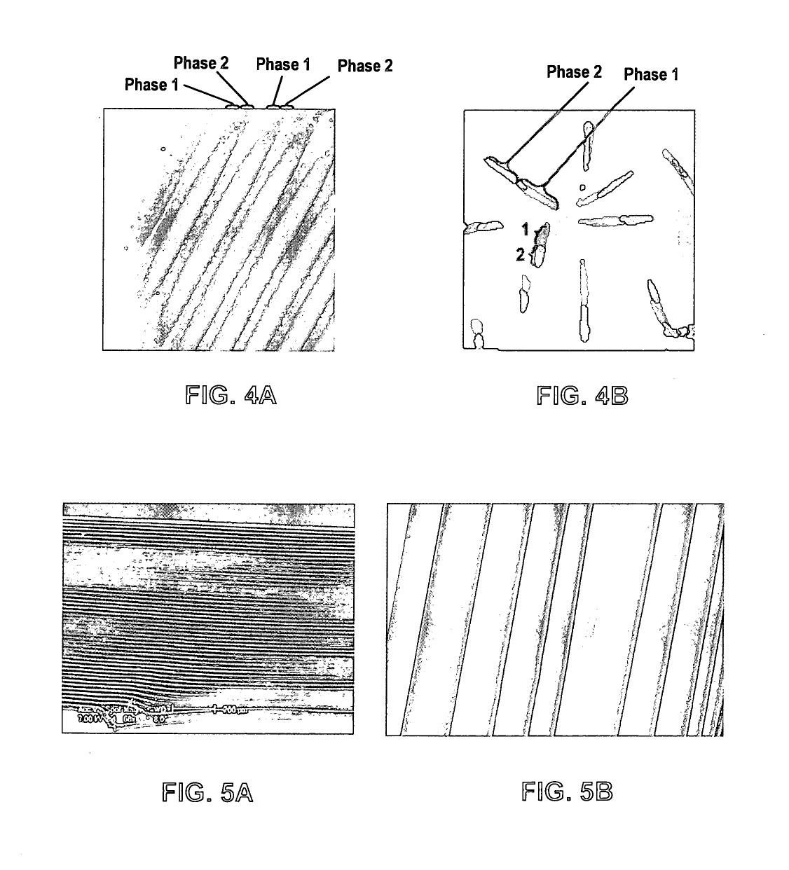

As further described in co-pending parent U.S. patent application Ser. No. 12/257,945 filed on Oct. 24, 2008 and entitled “Methods for Forming Biodegradable Nanocomponents With Controlled Shapes and Sized Via Electrified Jetting,” incorporated herein by reference, the various operating regimes for forming nanocomponent particle morphologies are described, including appropriate process parameters for forming fibers, among others. Where polymer concentration is lower and flow rates remain relatively low, discs are formed. On the other hand, when polymer concentration is increased and relatively high and a higher flow rate is selected, fibers are desirably formed. If flow rate is lower or intermediate for the same high concentration that forms fibers; spheres are formed instead. When relatively high flow rates are employed during electrojetting with an intermediate polymer concentration, rod or cylinder shapes are formed. In certain alternate aspects, the present disclosure further contemplates the tissue support comprising rod or cylinder shapes to support cell growth, cell proliferation, cell differentiation, cell repair, and/or cell regeneration. See e.g., FIG. 4B. Such rod or cylinder shaped nanocomponents can be used in combination with microfiber components or independently. Thus, the discussion herein with regard to features of the microfibers is also applicable to rod or cylinder components. Additionally, other micro-component or nano-component particles formed via electrohydrodynamic jetting may be used with microfibers discussed herein.

In accordance with various aspects of the present disclosure, the operating regime for the electrified jetting is preferably such that fibers are formed. In some aspects, the operating regime may be such that rods and cylinders are formed. As discussed above, while the behavior of a system during electrojetting is related to the compositions of the respective jetting fluids and can vary, generally fibers are formed by using relatively high polymer concentrations and relatively high flow rates during the electrified jetting process. Particularly significant variables to control during electrojetting in accordance with the present methods to create multiphasic nano-components having desired fiber morphology are the concentration of polymer in the liquid stream (and/or overall viscosity of the liquid stream based on all components present therein), as well as flow rate of the jetting liquid stream. Thus, in certain variations, multiphasic particles made in accordance with the inventive techniques are made with high shape selectivity for fibers.

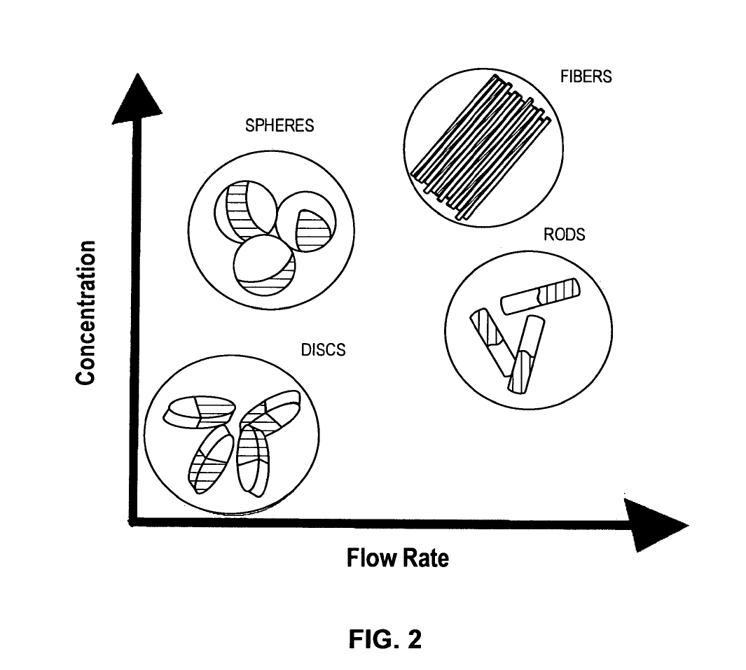

For example, in a system having PLGA polymer in the respective jetting solutions, the two process parameters which control nano-component shape are polymer concentration in solution and common (mixed stream) flow rate, as shown in FIG. 2. As can be seen in FIG. 2, electrified jetting at relatively low polymer (e.g., PLGA) concentrations and low flow rates produces discs, as where greater concentrations form spheres. At intermediate concentrations and higher flow rates, rods are formed, as where at higher concentration and higher flow rates, fibers are formed. As appreciated by those of skill in the art, the specific concentrations and flow rates that create the different morphologies may differ depending upon the polymer system selected, as well as other conditions during set-up, including temperature, pressure, humidity, so that the following values discussed in the context of the PLGA polymer are exemplary.

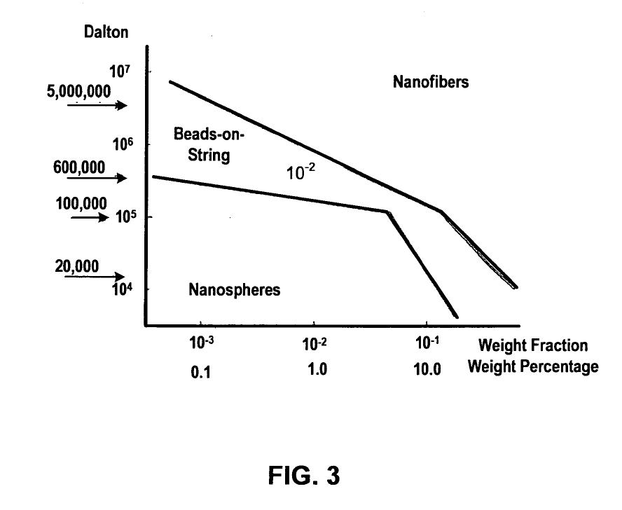

FIG. 3 demonstrates how nano-component morphology can be controlled by changing two independent solution parameters (concentration and molecular weight of the structural polymer). Here, use of higher polymer concentrations and larger molecules makes viscosity of the jetting solution higher so that the resulting morphology becomes more fibrous. For the same jetting liquids, use of different operating parameters also changes the resulting morphology. It is desirable to select these parameters for laminar flow of the jetting solutions during formation to form the multiphasic microfibers of the present disclosure.

In various aspects, the methods of forming such multiphasic microfiber components have a high selectivity (e.g., a high yield), which corresponds to forming greater than 50%, optionally at least about 70% of the plurality of nano-components so that they have substantially the same shape, size, and/or orientation of phases. Morphological control can be achieved with the exemplary electric jetting formation methods described herein. In various aspects, methods are provided which make a multiphasic microfiber component that includes forming a plurality of microfiber components by jetting two or more liquid streams together to form a mixed liquid stream that passes through an electric field generated by electrodes. The electric field is sufficient to form a cone jet that is capable of forming a plurality of microfiber components, each respectively having a first phase and at least one additional phase distinct from the first phase. Further, at least one of the phases comprises a biocompatible polymer and in certain variations, each phase comprises a biocompatible polymer. The forming of the plurality of microfiber components has a high selectivity with respect to at least one of: shape, size, and orientation of the first phase and/or the at least one additional phase. Such a high selectivity is achieved by controlling one or more of: polymer concentration in the liquid streams, flow rate of the mixed liquid stream, humidity, temperature, pressure, electrode design, and configuration of electrodes.

In certain aspects, shape selectivity can range from about 50 to 100%, optionally from about 70 to 99.5%, optionally about 85 to 99%, and in certain aspects, greater than or equal to about 90% up to 100% of the microcomponents formed have a fiber shape.

As recognized by those of skill in the art, while the general principles discussed herein apply, the concentration of polymer in a jetting solution and flow rate discussed below are exemplary for certain polymer systems and may vary based on the properties of the polymer and solvent employed. In certain embodiments, a combination of high polymer concentrations (e.g., 18:100 w/w polymer:solvent) and low flow rates (e.g., 0.02-0.04 ml/h), taken together with the low conductivities of organic solvents result in the formation of an extremely stable jet that yields a single fiber, with a well defined interface between individual jetting solutions in the droplet. Furthermore, by selecting a solution having lower conductivities and a forming a jet with a larger diameter, the temporal and spatial perturbations observed during conventional electrospinning processes are desirably minimized or eliminated. For higher polymer concentrations (e.g., 13 to 23% w/w of polylactide copolymer in chloroform containing 5 to 10% by vol. dimethyl formamide in each phase) in conjunction with lower flow rates (0.01-0.1 ml/h) well-defined multiphasic fibers are formed. During the cojetting, a single polymer jet comprising multiple streams is ejected from the cone tip. Higher polymer concentrations result in greater cone stability, as compared to co-jetting required for forming particles, and the Taylor cone can be uninterruptedly maintained for several hours. In fact, bending and whipping instabilities typically associated with electrojetting or electrospinning are widely suppressed under these conditions.

In various aspects, the present disclosure provides a process to prepare micro-structured scaffold materials made of multiphasic microfibers formed by electrohydrodynamic formation techniques described above. In various embodiments, flow rates and viscosities of at least two different polymeric solutions occurs through a set of capillaries to achieve laminar flow that creates multiphasic microfibers suitable for scaffolds or support structures.

As discussed above, the present methods also provide the ability to control phase alignment in the multiphasic microfibers. In certain aspects, biphasic fiber alignment is believed to result from a combination of controlling each jetting stream flow rate and polymer concentration. In certain conditions, anisotropic phase orientation is desirably controlled. The respective orientation of phases in the nano-component is also controlled during the formation process, so that in certain aspects, the phases are aligned along a major axis. In other aspects, two phases may be diametrically opposed to one another along a major axis of the microfiber or may form gradients along an axis of the microfiber or a rod shaped nanocomponent (as shown in FIG. 4B). In certain aspects, a plurality of nano-components formed in accordance with the present teachings have an axial geometry, such as a fiber shape, where each nano-component of the plurality has a longitudinal major axis so that respective longitudinal major axes of each of the plurality of nano-components are substantially aligned in a first orientation.

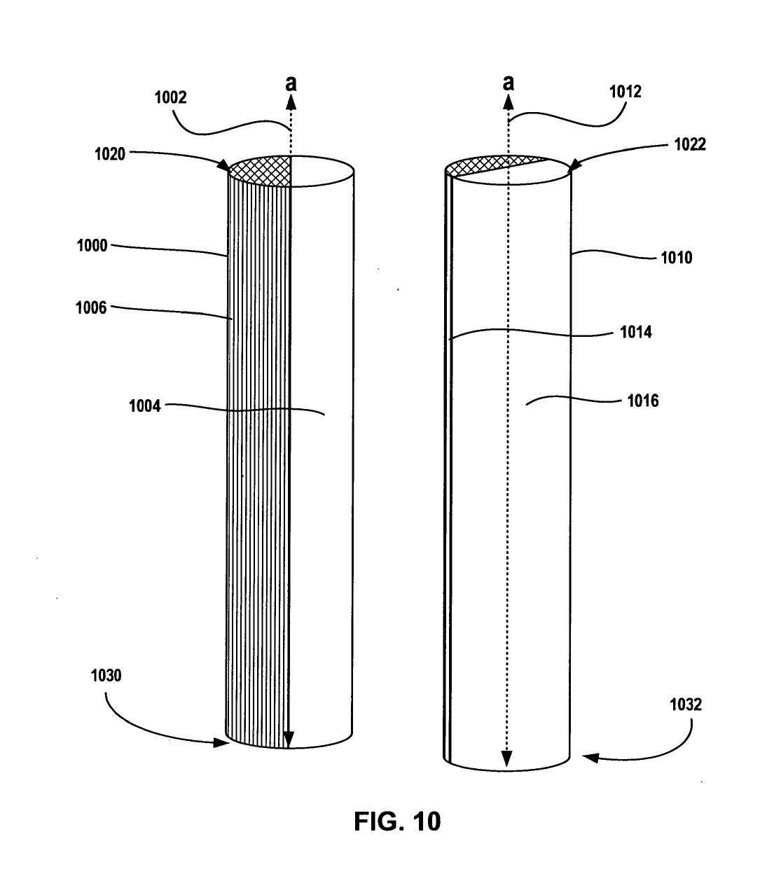

For example, in FIG. 10, a first biphasic microfiber 1000 has a longitudinal major axis “a” designated 1002 and two distinct phases, 1004, 1006, respectively. A second biphasic microfiber, 1010 has a longitudinal major axis “a” designated 1012 and two distinct phases, 1014, 1016. The respective phases 1004, 1006 of first microfiber 1000 and 1014, 1016 of second microfiber 1010 are substantially the same size and are aligned along the length of the longitudinal major axes “a” 1002, 1012 from a first terminus or end (1020 of first microfiber 1000 and 1030 of second microfiber 1010) to a second terminus or end (1022 of first microfiber 1000 and 1032 of second microfiber 1010). Thus, the first microfiber 1000 of a plurality of microfibers deposited in accordance with the present teachings defines the first longitudinal major axis 1002 and the second microfiber 1010 of the plurality defines a second longitudinal major axis 1012, wherein the first and second longitudinal major axes 1002, 1012 are substantially aligned with one another. See also FIGS. 4A, 5A-5B, 6A-6C and 6E-G.

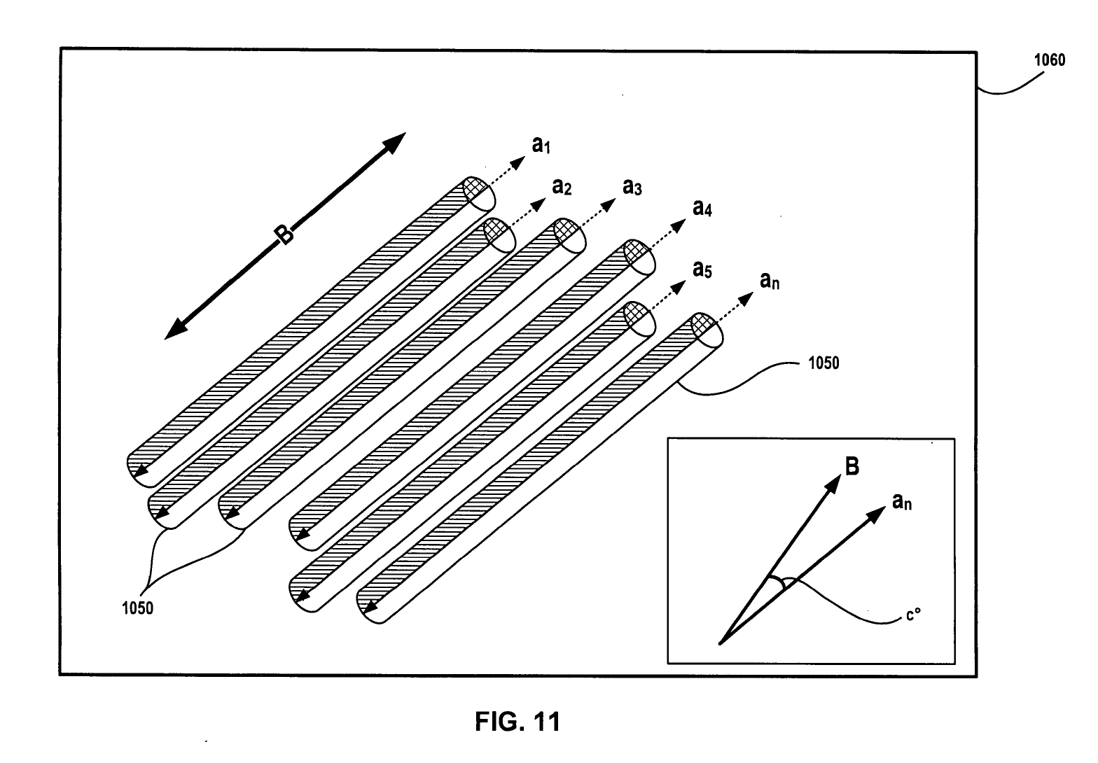

Further, as shown in FIG. 11, a plurality of microfibers 1050 are applied to a substrate plate 1060 by the electrohydrodynamic jetting methods described above. The plurality of axial geometry microfibers 1050 each define a major longitudinal axis respectively labeled “a₁-aₙ” in FIG. 11, which are applied to the substrate plate 1060 in such a manner that they are substantially aligned along a direction “B” on the substrate 1060. As shown in the inset, alignment of the microfibers can be expressed as deviating from direction “B” by measuring any angle “c” that occurs between the microfiber's major longitudinal axis an and B. Preferably, a plurality of microfibers are considered to be “substantially aligned” along a direction “B” when a maximum angle of deviation c for the orientation of the microfiber is less than or equal to about 20°, optionally less than or equal to about 15°, optionally less than or equal to about 10°, optionally less than or equal to about 7°, optionally less than or equal to about 5°, optionally less than or equal to about 4°, optionally less than or equal to about 3°, optionally less than or equal to about 2°, optionally less than or equal to about 1°, and in some aspects, 0°. Notably, the direction of the substrate 1060 may be modified so that the microfibers are applied as multiple different layers having different orientations or directions with respect to the substrate. See e.g., FIG. 1. The multi-directional assembly may deviate by a variety of angles, by way of non-limiting example, a layer formed by a plurality of substantially aligned microfibers may differ from an adjacent layer of a plurality of substantially aligned microfibers by 45°, 90°, 135°, 180°, 225°, 270°, or 315° to one another. Additionally, the substrate 1060 may be configured to be translated or rotated during the jetting process to generate a gradient of orientations of the plurality of microfibers during the electrohydrodynamic application process.

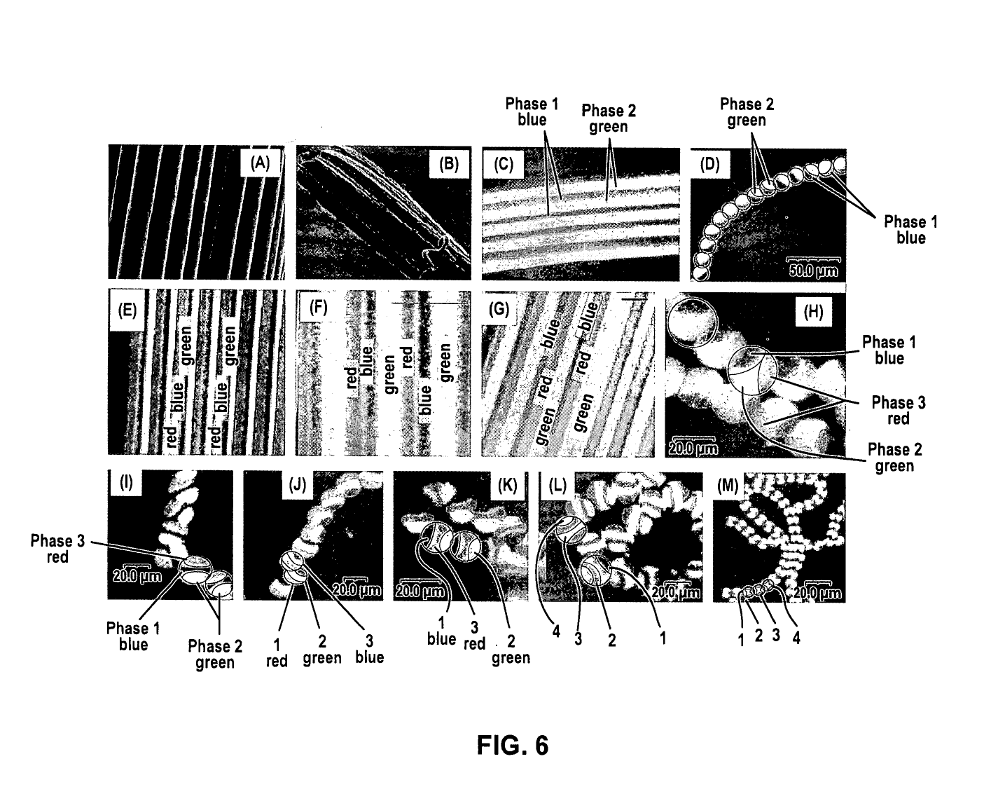

In certain embodiments, a first phase and at least one additional phase are also aligned in a first orientation along the major longtudinal axis for each microfiber component of the plurality, so that the microfibers themselves are aligned, as are the phases within the microfibers. See e.g., FIGS. 4A, 5A-5B, 6A-6C and 6E-G. Cross-sections of the plurality of microfibers are also shown for various examples, FIG. 6D shows a cross-section of the plurality of biphasic fibers in FIG. 6C, FIG. 6H is a cross-section of the triphasic fibers of FIG. 6E, the cross-sectional view of triphasic fibers of FIG. 6F is shown in FIG. 6J, and that of FIG. 6G in FIG. 6K. FIG. 6L shows a cross-section of tetraphasic fibers and FIG. 6K another cross-section of tetraphasic fibers having a diamond shaped orientation (formed by a diamond shaped orientation of needles for jetting). In yet other aspects, a plurality of microcomponents have a fiber or elongated rod shape where the first phase and at least one additional phase are diametrically opposed to one another along an axial direction of the microfiber. FIG. 4B.

Moreover, as noted above, the present disclosure further provides the ability to create aligned microfibers on a substrate, according to another aspect. Various embodiments provide a relatively simple technique to form aligned fibers, without requiring extensive additional setup or equipment. In certain aspects, the present disclosure provides a method of producing aligned nano-components in a fiber shape, based solely on manipulating solution properties that are being jetted; however, not requiring any other external changes in the jetting setup. Thus, where the components are in the form of fibers, a plurality of such microfibers can be formed having alternate alignment with respect to individual phases. In certain aspects, a plurality of micro-components are thus formed in accordance with the present teachings having a fiber shape, where each has a longitudinal major axis so that respective major axes of each of the plurality of nano-components are substantially aligned in a first orientation. See e.g., FIGS. 11 and 4A, 5A-5B, 6A-6M. In certain embodiments, a first phase and at least one additional phase are also aligned in a first orientation along the longitudinal major axis for each micro-component of the plurality, so that the fibers are aligned, as are the phases of the micro-components. Coupled with their unique, alternating phase alignment, the micro-component fibers provided by the present teachings have utility in a host of applications, including tissue engineering scaffolds, cell growth cultures, microfluidics, and the like, by way of non-limiting example.

While the methods described above having only an electric field or potential applied during electrohydrodynamic jetting desirably form microfibers, in alternate embodiments, additional control of the size of multiphasic nano- and microfibers can be achieved by superimposing the electrical field used for driving the electrohydrodynamic jetting with an oscillating field. Oscillating fields include, but are not limited to electric fields, mechanical fields, magnetic fields, or thermal pulses. Alternatively, a perturbation of the initial jet may be generated by jetting through a region with an oscillating electric field that deforms, interrupts, or deflexes the jet comprising the mixed liquid stream. This method can result in multiphasic micro- and nanofibers with monodisperse sizes. Monodisperse generally refers to size distributions of a species that have a standard deviation that is less than about 25%, optionally less than about 20%, optionally less than about 15%, optionally less than about 10%, optionally less than about 5%, and in some aspects, less than about 1% relative to the average of the size distribution of the species.

In various aspects, the present disclosure provides precisely engineered scaffolds or cellular support structures, which can be formed of multiphasic microfibers formed in accordance with the principles discussed above. For example, a scaffold structure can comprise microfibers formed from biocompatible non-degradable or biodegradable polymers, such as polymers, copolymers and combinations of a polylactic acid, polycaprolactone, and polyglycolic acid.

Thus, in various embodiments, the multiphasic microfiber comprises at least one biocompatible material, such as a biocompatible polymer. In certain aspects, multiple phases of the multiphasic microfiber each comprise one or more biocompatible materials, such as biocompatible polymers. By “biocompatible,” it is meant that a material or combination of materials can be contacted with cells, tissue in vitro or in vivo, or used with mammals or other organisms and has acceptable toxicological properties for contact and/or beneficial use with such cells, tissue, and/or animals. For instance, a biocompatible material may be one that is suitable for implantation into a subject without adverse consequences, for example, without substantial toxicity or acute or chronic inflammatory response and/or acute rejection of the material by the immune system, for instance, via a T-cell response. It will be recognized, of course, that “biocompatibility” is a relative term, and some degree of inflammatory and/or immune response is to be expected even for materials that are highly compatible with living tissue. However, non-biocompatible materials are typically those materials that are highly toxic, inflammatory and/or are acutely rejected by the immune system, e.g., a non-biocompatible material implanted into a subject may provoke an immune response in the subject that is severe enough such that the rejection of the material by the immune system cannot be adequately controlled, in some cases even with the use of immunosuppressant drugs, and often can be of a degree such that the material must be removed from the subject. In certain aspects, biocompatible materials are those that are approved for use in humans by an appropriate regulatory agency, such as the Federal Drug Administration (FDA) in the United States; the European Commission (EC)/European Medicines Agency (EMEA) in Europe; or Health Products and Food Branch (HPFB) in Canada.

Thus, multiphasic microfibers can be made of a wide variety of materials, including inorganic and organic biocompatible materials. Specifically, biocompatible polymer materials, such as biodegradable or non-biodegradable polymers, synthetic or natural polymers can be used to form the microfiber components. In one aspect, the first phase of the multiphasic microfiber comprises a first biocompatible polymer and the second phase comprises a second biocompatible polymer that is distinct from the first polymer. Further, each phase may comprise a plurality of different materials, such as a plurality of different biocompatible polymers. Thus, in certain aspects different polymers can be used in at least two phases of the multiphasic microfiber composition.

In certain aspects, the polymers can also be modified by chemical or physical methods, such as cross-linking, heat treatment, photochemical treatment, and/or changes in the chemical or physical environment. In a certain aspects, the polymer modification occurs in a select portion or region of one or more of the multiple phases of the microfiber, or such polymer modification can occur to different degrees, potentially resulting in different materials or materials responses, as appreciated by one of skill in the art. Such polymer modification and/or treatment provides different release kinetics in certain aspects. Further, surface alterations, such as differences in hydrophilicity, charge, or other physical properties, facilitate cell adhesion.

In certain respects, different polymers used in the different phases of the microfiber permit different active ingredient release kinetics, different surface properties, or surfaces having different moieties exposed, which can be useful in designing spatially guided cellular growth and in certain aspects to facilitate adhesion of cells or tissue or to promote release of biofunctional agents, which include biofunctional materials and biofunctional active ingredients (e.g., pharmaceutical active ingredients), and the like, into the surrounding environment. Further, otherwise incompatible ingredients can be delivered simultaneously to a target region by employing two or more distinct polymer phases in a single microfiber.

One phase may contain a first biofunctional active ingredient and a second phase may contain a second biofunctional active ingredient that is otherwise incompatible with the first active ingredient. The first phase comprises material(s) compatible with the first component and the second phase similarly has material(s) compatible with the second component. Thus, a lipophilic or hydrophobic biofunctional active ingredient can be included in one phase of the multiphasic microfiber and a hydrophilic biofunctional active ingredient can be included in a second phase, however both the first and second active ingredients are delivered and bioavailable to target cells or tissues. Similarly, a cationic biofunctional active ingredient can be contained in a first phase of the multiphasic microfiber and an anionic biofunctional active ingredient can be contained in a second phase of the multiphasic microfiber to provide localized availability of both cationic and anionic active ingredients concurrently to the target cells or surrounding tissue.

In certain aspects, certain phases of the multiphasic microfiber dissolve or disintegrate at different rates ex vivo or in vivo. In this regard, the dissolution rate of the respective phases impacts the release rate of biofunctional substances and/or active ingredients from each phase, thus providing control over the release kinetics and concentration of biofunctional substances and active ingredients to be delivered to target regions in the local environment from each respective phase of the nano-component. As referred to herein, “dissolve” refers to physical disintegration, erosion, disruption and/or dissolution of a material and may include the resorption of a material by a living organism. The phases may dissolve or disintegrate at different rates or have different solubility (e.g., aqueous solubility) that impacts the rate of biofunctional active ingredient release. In certain variations, each phase comprises one or more materials that dissolve or erode upon exposure to a solvent comprising a high concentration of water, such as serum, growth or culture media, blood, bodily fluids, or saliva. In some variations, a phase may disintegrate into small pieces or may disintegrate to collectively form a colloid or gel. In some aspects, a phase of the multiphasic microfiber comprises a polymer that is insoluble or has limited solubility in water, but is dispersible in water, so that the polymer breaks down or erodes into small fragments. In other aspects, a polymer used in a phase of the multiphasic microfiber is insoluble in water, but may be swellable. In variations where a polymer does not fully break down, the polymer can be a water-repellant polymer or an aqueous-stable hydrophilic polymer, for example, certain types of cellulose. For structural scaffold members, the dissolution rate (e.g., a rate at which the structural member is resorbed by surrounding cells) can be designed so that sufficient cellular growth occurs prior to the structure dissolving or disintegrating through the resorption process. In various aspects, each phase of the multiphasic microfiber optionally comprises a combination of biocompatible polymer materials.

Particularly suitable non-limiting polymers for use in the multiphasic compositions (e.g., in Fluid A designated 100 or Fluid B designated 102 of FIG. 1) include sodium polystyrene sulfonate (PSS), polyethers, such as a polyethylene oxide (PEO), polyoxyethylene glycol or polyethylene glycol (PEG), polyethylene imine (PEI), a biodegradable polymer such as a polylactic acid, polycaprolactone, polyglycolic acid, poly(lactide-co-glycolide polymer (PLGA), and copolymers, derivatives, and mixtures thereof. Other polymers discussed herein include those well known to those of skill in the art to be used in cell cultures, implants, regenerative, therapeutic, and pharmaceutical compositions. One such example is polyvinylpyrrolidone (PVP).

Specifically, at least one phase can be designed to have one or more of the following properties based upon material selection: hydrophobic, positively-charged (cationic), negatively-charged (anionic), polyethylene glycol (PEG)-ylated, covered with a zwitterion, hydrophobic, superhydrophobic (for example having with water contact angles in excess of 150°), hydrophilic, superhydrophilic (for example, where the water contact angle is near or at 0°), olephobic/lipophobic, olephilic/lipophilic, and/or nanostructured, among others.

In other aspects, one or more polymers or materials used within a phase may be functionalized to subsequently undergo reaction with various moieties or substances after formation of the multiphasic nano-component, to provide desired surface properties or to contain various moieties presented on the phase surface (e.g., for surface patterning), as recognized by those of skill in the art.

Water-soluble and/or hydrophilic polymers, which are biocompatible, include cellulose ether polymers, including those selected from the group consisting of hydroxyl alkyl cellulose, including hydroxypropyl methyl cellulose (HPMC), hydroxypropyl cellulose (HPC), hydroxyethyl cellulose (HEC), methyl cellulose (MC), carboxymethyl cellulose (CMC), and mixtures thereof. Other polymers among those useful herein include polyvinylpyrrolidone, vinyl acetate, polyvinylpyrrolidone-vinyl acetate copolymers, polyvinyl alcohol (PVA), acrylates and polyacrylic acid (PAA), including polyacrylate polymer, vinylcaprolactam/sodium acrylate polymers, methacrylates, poly(acryl amide-co-acrylic acid) (PAAm-co-AA), vinyl acetate and crotonic acid copolymers, polyacrylamide, polyethylene phosphonate, polybutene phosphonate, polystyrene, polyvinylphosphonates, polyalkylenes, and carboxy vinyl polymer. The multiphasic fiber compositions may comprise derivatives, copolymers, and further combinations of such polymers, as well.

Other polymers or water-soluble fillers among those useful herein include, without limitation, sodium alginate, carrageenan, xanthan gum, gum acacia, Arabic gum, guar gum, pullulan, agar, chitin, chitosan, pectin, karaya gum, locust bean gum, various polysaccharides; starches such as maltodextrin, amylose, corn starch, potato starch, rice starch, tapioca starch, pea starch, sweet potato starch, barley starch, wheat starch, modified starch (e.g., hydroxypropylated high amylose starch), dextrin, levan, elsinan and gluten; and proteins such as collagen, whey protein isolate, casein, milk protein, soy protein, keratin, and gelatin.

Further, non-limiting examples of water insoluble or hydrophobic polymers include cellulose acetate, cellulose nitrate, ethylene-vinyl acetate copolymers, vinyl acetate homopolymer, ethyl cellulose, butyl cellulose, isopropyl cellulose, shellac, hydrophobic silicone polymer (e.g., dimethylsilicone), polymethyl methacrylate (PMMA), cellulose acetate phthalate and natural or synthetic rubber; siloxanes, such as polydimethylsiloxane (PMDS), polymers insoluble in organic solvents, such as cellulose, polyethylene, polypropylene, polyesters, polyurethane and nylon, including copolymers, derivatives, and combinations thereof. The polymers may be crosslinked after formation by application of heat, actinic radiation or other methods of curing and treating polymers known to those of skill in the art. Additionally, in certain aspects, other synthetic and natural biocompatible polymers known or to be discovered in the art are contemplated by alternate variations of the present disclosure.

In various aspects of the present disclosure, the polymers are present in a liquid phase prior to electrified jetting or spraying at about 0.1 to about 100% by weight (on a wet basis). While the relative concentrations of polymers in a phase can vary greatly depending on the polymer, application, and process parameters used for forming the nano-component, in certain aspects, the polymer is optionally present at about 5% to about 50% by weight; optionally from about 7% to about 20% by weight of the phase; optionally about 10% to about 20%; optionally about 16% to about 20% by weight of the phase.

Multiphasic microfibers formed in accordance with the methods of the present disclosure may have in some (but not necessarily all) embodiments, one or more of the following advantages:

1) that the number of phases (e.g., compartments) within each microfiber can be tailored to be from two to more than a hundred;

2) respective phase spatial orientation is highly controlled and degree of phase alignment within each fiber is highly reproducible;

3) a plurality of microfibers formed by the methods of the present disclosure are themselves well aligned to facilitate ready formation of support substrates and tissue scaffolds;

4) the respective phases are designed to have distinct biofunctionality or physical properties when interacting with the external surrounding environment (e.g., surrounding cells or tissue). The presence of distinct exposed phases enables selective surface patterning with biological ligands, such as for example, peptides like a laminin-derived IKVAV-containing (pentapeptide sequence of Ile-Lys-Val-Ala-Val) or other cell adhesion peptides, growth factors, antibodies, sugars, nucleic acids, such as DNA and RNA, and the like. For example, one phase may react with cell adhesion ligands that covalently bind peptides or cells to only certain phase(s) of the fibers;

5) ability to selectively design phases for targeted cellular growth or inhibition (e.g., fibroblasts can be cultured on a selectively peptide-modified phase of a microfiber forming a part of a scaffold having cells adhering and growing along only the peptide-containing phase or compartment). Human, mammalian, or non-mammalian cells may be cultured on selectively modified scaffolds in a two- or three-dimensional setting. Specific examples of cells that can be proliferated in such a manner include by way of non-limiting example, fibroblasts, endothelial cells, hepatocytes, epithelial cells, stem cells, human embryonic stem cells, neurons, neuronal progenitor cells; and

6) the multiphasic fibers of the present disclosure can be used for culturing a single cell type or multiple different cell types. If a co-culture of multiple cell types is desired, the individual phases may be designed to selectively attract or repel different types of cells. In various aspects, the microfibers made in accordance with the present teachings can be used to create precisely-engineered three-dimensional biocompatible microfiber scaffolds. Thus, scaffolds formed from the multiphasic microfibers of the present disclosure offer spatial and directional control over cell growth and proliferation.

In various aspects, the microfibers of the present disclosure support and/or promote cell growth, proliferation, differentiation, regeneration, and/or repair, for example. By “promoting” cell growth, cell proliferation, cell differentiation, cell repair, or cell regeneration, it is meant that a detectable increase occurs in either a rate or a measurable outcome of such processes in the presence of the microfiber as compared to a cell or organism's process in the absence of the microfiber, for example, conducting such processes naturally. By way of example, as appreciated by those of skill in the art promoting cell growth in the presence of the microfiber may increase a growth rate of target cells or increase a total cell count of the target cells, when compared to cell growth or cell count of the target cells in the absence of such microfiber. By “supporting” cell growth, cell proliferation, cell differentiation, cell repair, or cell regeneration, it is meant that the microfiber provides a physical substrate for one or more target cells that enhances target cell growth, vitality, proliferation, differentiation, repair, or regeneration, by way of non-limiting example. As appreciated by those of skill in the art, the microfiber may both support and promote the growth, vitality, proliferation, differentiation, repair, and/or regeneration processes of one or more target cells in vitro, ex vivo, or in vivo, for example. In certain variations, two of more microfibers are selected to form a cellular scaffold structure that supports and/or promotes target cell growth, target cell proliferation, target cell differentiation, target cell repair, and/or target cell regeneration in three-dimensions, in contrast to the support and growth on conventional two-dimensional planar surfaces. The distinct multiphasic microfibers of the present disclosure can be employed to create a scaffold design that promotes growth of one or more target cells in a predetermined three-dimensional pattern.

In certain aspects, one or more phase surfaces of the microfiber interact with a target (e.g., a target cell) in the surrounding environment to promote such an effect or outcome (cell growth, proliferation, differentiation, regeneration, and/or repair). For example, in certain embodiments, the material selection of a phase, surface treatment of a phase surface, or the structure, configuration, gradient, and/or orientation of distinct phases in the microfiber may promote such a desirable outcome. As described further below, certain phases may further comprise at least one biofunctional agent capable of interacting with the surrounding environment to promote or enhance such an effect, as well.

Various ways to qualitatively or quantitatively measure cell growth, cell proliferation, cell differentiation, cell repair, or cell regeneration are well known in the art and often vary depending upon the target cells of interest. Generally, cell proliferation assays, high-throughput screening, high-content screening (HCS), and a range of other assay formats, such as fluorescent, luminescent, and colorimetric assays, provide imaging or measurement of cell function, metabolism, and signaling. Such assays can optionally measure cell proliferation, determine cell growth by total cell count, live versus dead cell count, detect DNA synthesis, measure metabolic activity or proliferative activity, and the like.

By way of non-limiting further example, cell viability can be measured by cell counts or metabolic activity assays. Cell proliferation can be measured by clonogenic assays and colony formation, or measuring DNA synthesis with a proliferation marker, or measuring cell cycle regulators by activity level (e.g., CDK kinase assays) or quantifying the amount of a signaling molecule present (e.g., Western blots, ELISA, and the like), by way of non-limiting example. Cell differentiation can be measured by evaluating cell morphology changes or outgrowth or measuring a level of genes or other expression markers generated during differentiation (e.g., a level of specific complement receptors or other proteins or cellular expression markers via a Western blot), by way of non-limiting example. Cell repair may be measured by quantitative or quantitative measurement of DNA damage/repair levels, for example, in a single-cell gel electrophoresis assay (“comet assay”).

In certain variations, the microfiber's interaction with a surrounding environment promotes cell growth, cell proliferation, cell differentiation, cell repair, and/or cell regeneration by increasing a measurable process result (e.g., measuring the rates, quantitative or qualitative results for cell generation, cell regeneration, cell vitality, cell proliferation, cell differentiation, or cell repair rates) by greater than or equal to about 25% as compared to the result of the process in the absence of the microfiber, optionally increasing by greater than or equal to about 30%, optionally increasing by greater than or equal to about 35%, optionally increasing by greater than or equal to about 40%, optionally increasing by greater than or equal to about 45%, optionally increasing by greater than or equal to about 50%, optionally increasing by greater than or equal to about 55%, optionally increasing by greater than or equal to about 60%, optionally increasing by greater than or equal to about 65%, optionally increasing by greater than or equal to about 70%, optionally increasing by greater than or equal to about 75%, optionally increasing by greater than or equal to about 80%, optionally increasing by greater than or equal to about 85%, optionally increasing by greater than or equal to about 90%, and in certain aspects, optionally increasing by greater than or equal to about 95%.

Similarly, in other aspects, the microfiber promotes a rate of cell growth, cell proliferation, cell differentiation, cell repair, and/or cell regeneration by enhancing a rate of the desired process by greater than or equal to about 25% as compared to the rate of the process in the absence of the microfiber, optionally greater than or equal to about 30%, optionally greater than or equal to about 35%, optionally greater than or equal to about 40%, optionally greater than or equal to about 45%, optionally greater than or equal to about 50%, optionally greater than or equal to about 55%, optionally greater than or equal to about 60%, optionally greater than or equal to about 65%, optionally greater than or equal to about 70%, optionally greater than or equal to about 75%, optionally greater than or equal to about 80%, optionally greater than or equal to about 90%, and in certain aspects, optionally greater than or equal to about 95%.

In certain aspects, one or more of the phases of the multiphasic microfibers comprises a “biofunctional” agent, which refers to a material or chemical substance, such as a small molecule, active ingredient, macromolecule, ligand, metal ion, or the like, that is bioactive and causes an observable change in the structure, function, optical function, or composition of a target cell, when such a target cell is exposed to such a material or substance. The term “biofunctional agent” and “biofunctional active ingredient” are used interchangeably herein. Non-limiting examples of observable cellular changes include increased or decreased expression of one or more mRNAs, DNA, or other nucleotides, increased or decreased expression of one or more proteins, phosphorylation of a protein or other cell component, inhibition or activation of an enzyme, inhibition or activation of binding between members of a binding pair, an increased or decreased rate of synthesis of a metabolite, increased or decreased generation of immune system cells, hormones, growth factors, or other intercellular mediators and signaling agents, increased or decreased cell proliferation, enhanced cellular growth, such as germline or somatic cell growth, changes in optical properties, and the like. In various aspects of the present disclosure, the biofunctional agent promotes cellular development affecting cell shape, size, proliferation, growth, death, motility, state of differentiation, interaction with other cells, interaction with extracellular materials, or transcriptional, translational, or metabolic profile. In certain aspects, the multiphasic microfibers of the disclosure deliver active ingredients to a target, which in some embodiments is to cells, tissue or to an organ of an organism.

In various aspects, the biofunctional agent present in the microfibers of the present disclosure promotes cell regeneration, differentiation, growth, proliferation, and/or repair, for example. By “promoting” cell growth, cell proliferation, cell differentiation, cell repair, or cell regeneration, it is meant that a detectable increase occurs in either a rate or a measurable outcome of such processes occurs in the presence of the biofunctional agent as compared to a cell or organism's process in the absence of such a biofunctional agent, for example, conducting such processes naturally, as discussed previously above. By way of example, as appreciated by those of skill in the art promoting cell growth in the presence of a biofunctional agent may increase a growth rate of target cells or increase a total cell count of the target cells, when compared to cell growth or cell count of the target cells in the absence of such a biofunctional agent.

In certain variations, the biofunctional agent promotes cell growth, cell proliferation, cell differentiation, cell repair, and/or cell regeneration by increasing a measurable process result (e.g., measuring total cell counts for cell generation or cell regeneration, measuring the rates or qualitative outcome of cell proliferation, cell differentiation, or cell repair rates) by greater than or equal to about 25% as compared to the result of the process in the absence of the biofunctional agent, optionally increasing by greater than or equal to about 30%, optionally increasing by greater than or equal to about 35%, optionally increasing by greater than or equal to about 40%, optionally increasing by greater than or equal to about 45%, optionally increasing by greater than or equal to about 50%, optionally increasing by greater than or equal to about 55%, optionally increasing by greater than or equal to about 60%, optionally increasing by greater than or equal to about 65%, optionally increasing by greater than or equal to about 70%, optionally increasing by greater than or equal to about 75%, optionally increasing by greater than or equal to about 80%, optionally increasing by greater than or equal to about 85%, optionally increasing by greater than or equal to about 90%, and in certain aspects, optionally increasing by greater than or equal to about 95%.

Similarly, in other aspects, the biofunctional agent promotes cell growth, cell proliferation, cell differentiation, cell repair, and/or cell regeneration by enhancing the rate of the desired process by greater than or equal to about 25% as compared to the rate of the process in the absence of the biofunctional agent, optionally greater than or equal to about 30%, optionally greater than or equal to about 35%, optionally greater than or equal to about 40%, optionally greater than or equal to about 45%, optionally greater than or equal to about 50%, optionally greater than or equal to about 55%, optionally greater than or equal to about 60%, optionally greater than or equal to about 65%, optionally greater than or equal to about 70%, optionally greater than or equal to about 75%, optionally greater than or equal to about 80%, optionally greater than or equal to about 85%, optionally greater than or equal to about 90%, and in certain aspects, optionally greater than or equal to about 95%.

In certain variations, at least one phase of the multiphasic nano-component comprise a biofunctional active ingredient that is a pharmaceutically active ingredient, which refers to a material or combination of materials that are used with mammals or other organisms having acceptable toxicological properties for beneficial use with such an animal. By way of non-limiting example, the biofunctional agent/active ingredient included in one or more phases of a multiphasic microfiber can be a therapeutic drug that operates locally or systemically (non-localized) and may treat, prevent, or diagnose a wide variety of conditions or ailments. In certain aspects, such active ingredients can be provided in one or more phases of a tissue scaffold implant to provide benefits in vivo.

Thus, such a biofunctional active ingredient is a compound or composition that diagnoses, prevents, or treats a physiological or psychological disorder or condition, or can provide a therapeutic, regenerative, cosmetic or aesthetic benefit in an organism, such as an animal, e.g., a mammal like a human. In certain aspects, a pharmaceutically active ingredient prevents or treats a disease, disorder, or condition of hard or soft tissue in an organism, such as a mammal. For example, a biofunctional agent can be targeted to a particular region in the mammal, such as organs, tissues, medical implants or devices, skeletal system, hair, skin, mouth, eyes, circulatory system, and the like.

Hence, the multiphasic microfibers may further comprise a biofunctional agent or material useful for repairing, regenerating or strengthening tissues. Such biofunctional or bioactive materials may be disposed on a surface of one or more phases of the multiphasic microfibers or may be distributed throughout (e.g., homogeneously mixed) the material forming the phase (and thus, may be exposed at the surface, as well). Biofunctional agents encompass therapeutic agents, such as pharmaceutically active agents, like drugs, and also genetic materials and biological materials. It should be appreciated that any agent discussed in the context of the present disclosure may have efficacy in several categories of an active agent and a discussion or listing of such an active agent under a given category is not exclusive or limiting of the active agent's utility.

Genetic materials encompass without limitation nucleotides or nucleic acids intended to be inserted into a human body, including viral vectors and non-viral vectors. Biofunctional agents include cells, yeasts, bacteria, proteins, peptides, cytokines and hormones, naturally occurring growth factors; proteins, peptides, peptoids, and small molecules identified by selection from chemical libraries, by way of non-limiting example.

Thus, in certain variations, the biofunctional agent is a pharmaceutically active composition. Pharmaceutically active compositions include drug and therapeutic compositions, oral care compositions, nutritional compositions, personal care compositions, cosmetic compositions, diagnostic compositions, and the like. In certain aspects, the pharmaceutically active composition is used in the multiphasic microfibers to form medical devices and implants, including tissue scaffolds, or can be provided as surface films or coatings for such apparatuses. In various aspects, the multiphasic microfibers may be used in a wide variety of different biological applications and may have other biofunctional agents, and are not limited those described herein. However, the present disclosure contemplates multiphasic microfibers comprising one or more biofunctional agents that provide a diagnostic, therapeutic, prophylactic, cosmetic, sensory, and/or aesthetic benefit to an organism, such as a mammal like a human. In certain aspects, the multiphasic microfibers optionally comprise one or more biofunctional agents, which optionally may be provided in a biocompatible composition in the respective phases of the microfibers.

In certain variations, the multiphasic microfibers of the present disclosure can be used in exemplary medical implants, such as cellular scaffolds or grafts, stem tissue scaffolds or grafts, tissue scaffolds and grafts, organ scaffolds or grafts and/or transplants, appendage scaffolds or grafts, genetic therapy or stem cell therapy, among others. In various aspects, where a multiphasic microfiber is used in an implant or in conjunction with a medical device or transplant, a variety of biofunctional agents/active ingredients can be employed to promote healing, such as promoting growth and reducing inflammation. A cellular graft is selected from the group consisting of a stem cell graft, a progenitor cell graft, a hematopoietic cell graft, an embryonic cell graft and a nerve cell graft, by way of non-limiting example. Exemplary tissue scaffolds or grafts are selected from the group consisting of skin, bone, nerve, intestine, corneal, cartilage, cardiac tissue, cardiac valve, dental, hair follicle, muscle, and the like. Organ scaffolds or grafts are selected from the group consisting of a kidney scaffold or graft, a heart scaffold or graft, a skin scaffold or graft, a liver scaffold or graft, a pancreatic scaffold or graft, a lung scaffold or graft and an intestine scaffold or graft, by way of non-limiting example. Suitable appendages are selected from the group consisting of a shin scaffold or graft, an arm scaffold or graft, a leg scaffold or graft, a hand scaffold or graft, a foot scaffold or graft, a finger scaffold or graft, a toe scaffold or graft, and the like. In certain aspects, the tissue scaffold or medical implant comprising the multiphasic microfiber(s) treats defective, diseased, damaged, and/or ischemic cells, tissue, or organ(s). Further, multiphasic microfibers having such biofunctional active ingredients can be used in conjunction with wound dressings, gauze, films, and the like.

Thus, biofunctional/active ingredients may be used to repair or regenerate cells of an organ or tissue; treat or prevent a disease, such as an infectious disease (a bacterial, viral, or fungal infection) or a degenerative disease (Alzheimer's, amyotrophic lateral sclerosis (ALS)). For example, active ingredients may treat an auto-immune disorder (e.g., rheumatoid arthritis, systemic lupus erythematosus (SLE), inflammatory bowel disease (IBD)), allergies, asthma, osteoarthritis, osteoporosis, cancer, diabetes, arteriosclerosis and cardiovascular disease, stroke, seizures, psychological disorders, pain, acne, caries, gingivitis, periodontitis, an H₂ antagonist, human immunodeficiency, infections, and the like. Particularly suitable biofunctional active agents for multiphasic microfibers used in implants and for tissue scaffolds include agents used to minimize an organism's immune response to foreign matter (e.g., to reduce host rejection), to reduce thrombosis and clotting, to reduce pain, infection, and inflammation, to promote adhesion of certain target cells, to promote healing, cellular repair, and growth, and to promote tissue differentiation and proliferation, by way of non-limiting example.

The description of suitable biofunctional agents/active ingredients is merely exemplary and should not be considered as limiting as to the scope of biofunctional active ingredients which can be introduced into the multiphasic microfibers according to the present disclosure, as all suitable biofunctional agents and/or active ingredients known to those of skill in the art for these various types of compositions are contemplated. Furthermore, a biofunctional agent/active ingredient may have various functionalities and thus, can be listed in an exemplary class below; however, may be categorized in several different classes of active ingredients. Various suitable active ingredients are disclosed in Merck Index, An Encyclopedia of Chemicals, Drugs, and Biologicals, Thirteenth Edition (2001) by Merck Research Laboratories and the International Cosmetic Ingredient Dictionary and Handbook, Tenth Ed., 2004 by Cosmetic Toiletry and Fragrance Association, and U.S. Pat. Nos. 6,589,562, 6,825,161, 6,063,365, and 6,491,902, all to Shefer et al, each incorporated herein by reference. Each additional reference cited or described herein is hereby expressly incorporated by reference in its respective entirety.

More specifically, suitable biofunctional agents include by way of non-limiting example, growth factors; growth factor receptors; transcriptional activators; translational promoters; anti-proliferative agents; growth hormones; anti-rejection drugs; anti-thrombotic agents; anti-coagulants; stem cell or gene therapies; antioxidants; free radical scavengers; nutrients; co-enzymes; ligands; cell adhesion peptides; peptides; proteins; nucleic acids; DNA; RNA; polysaccharides; sugars; nutrients; hormones; antibodies; immunomodulating agents; growth factor inhibitors; growth factor receptor antagonists; transcriptional repressors; translational repressors; replication inhibitors; inhibitory antibodies; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); COX-I and II inhibitors; antimicrobial agents; antiviral agents; antifungal agents; antibiotics; anti-proliferative agents; antineoplastic/antiproliferative/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; aptamers; quantum dots; nano-materials; nano-crystals; and combinations thereof.

In various aspects, one or more exposed phase surfaces of the microfibers comprise a biofunctional agent or moiety. In certain aspects, the presence of distinct exposed phases enables selective surface patterning with biofunctional agents, such as ligands, peptides (particularly cell adhesion peptides), cell adhesion molecules, proteins, nucleic acids, growth factors, hormones, antibodies, sugars, saccharides, nutrients, and the like. In certain aspects, the moiety may be provided to interact with the surrounding environment (for example, to avoid detection by an immune system, provide optical properties to the multiphasic microfiber, provide binding to a biological or non-biological target, such as cells or tissue or a medical device). In some aspects, the moiety is a binding moiety that provides the ability for the multiphasic microfiber to bind with a target. In certain aspects, the target may be a cell of an organism, such as germline or somatic cells, protein, enzyme, immune system cells, or other circulating cells or substances associated with the animal. The following discussion provides non-limiting examples of suitable binding moieties for use with the multiphasic microfiber components of the disclosure.

Suitable biofunctional agents, which may optionally be surface bound moieties on one or more phases of the multiphasic microfiber can be a growth factor. Many transforming growth factors (TGF-β super family) can be used for a wide range of therapeutic treatments and applications, which in particular, pertain to promotion of cell proliferation and tissue formation, including wound healing, tissue reproduction, and tissue regeneration. Such growth factors in particular include members of the TGF-β family, like the DVR-group, including bone morphogenetic protein (BMPs), growth differentiation factors (GDFs), inhibin/activin, and the GDNF protein family, by way of non-limiting example, as will be described in greater detail below. Often individual growth factors of these families exhibit multiple functions concurrently, so that they can be of interest for various treatment modalities. Some of these multifunctional proteins also have survival promoting effects on neurons in addition to functions such as regulation of the proliferation and differentiation in many cell types.

Furthermore, the multiphasic microfibers optionally comprise biofunctional agents that inhibit growth or response of certain targeted tissues, for example, cancer or immune system cells. In certain aspects, the multiphasic microfibers have one phase comprising a biofunctional agent to promote growth, proliferation, differentiation and/or repair of certain target cells, while another distinct biofunctional agent may inhibit growth of distinct target cells. For example, a multiphasic microfiber optionally includes growth factors, growth factor receptors, transcriptional activators, and translational promoters for promoting cell growth and may further optionally include cell growth inhibitors such as antiproliferative agents, growth factor inhibitors, growth factor receptor antagonists, transcriptional repressors, translational repressors, replication inhibitors, inhibitory antibodies, antibodies directed against growth factors, biofunctional molecules consisting of a growth factor and a cytotoxin, biofunctional molecules consisting of an antibody and a cytotoxin, and the like.

In certain variations, examples of the biofunctional agents include, but are not limited to, peptides and proteins, including erythropoietin (EPO), stem cell factor (SCF), vascular endothelial growth factor (VEGF), keratinocyte transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factor (CGF), stem cell factor (SCF), platelet-derived growth factor (PDGF), endothelial cell growth supplement (EGGS), colony stimulating factor (CSF), growth differentiation factor (GDF), integrin modulating factor (IMF), calmodulin (CaM), thymidine kinase (TK), tumor necrosis factor (TNF), growth hormone (GH), cartilage transcription factor SRY-related HMG-box gene 9 (Sox-9), bone morphogenic proteins (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, and the like), interferon, interleukins (e.g., IL-1, IL-2, IL-3, IL-4, IL-5, IL-6, IL-7, IL-8, IL-9, IL-10, IL-11, IL-12, IL-15, and the like), cytokines, integrins, selectins, cadherins, collagen, elastin, fibrillins, nectins (e.g., fibronectin, hemonectin), laminin, glycosaminoglycans, vitronectin, thrombospondin, heparan sulfate, dermantan, chondrotin sulfate (CS), hyaluronic acid (HA), proteoglycans, transferrin, cytotactin, tenascin, lymphokines, cell adhesion molecules, (e.g., neural cell adhesion molecules (N-CAMS), intercellular cell adhesion molecules (ICAMS), vascular cell adhesion molecules (VCAM), platelet-endothelial cell adhesion molecules (PECAM)).

Cell adhesion peptides are also particularly suitable biofunctional agents, particularly for exposure via an exposed phase surface, such as laminin derived IKVAV (Ile-Lys-Val-Ala-Val) and YIGSR peptide (Tyr-Ile-Gly-Ser-Arg), fibronectin derived RAD peptide (Arg-Gly-Asp), RGDS peptide (Arg-Gly-Asp-Ser), RGES peptide (Arg-Gly-Glu-Ser), EILDV peptide (Glu-Ile-Leu-Asp-Val), EILEVPST peptide (Glu-Ile-Leu-Glu-Val-Pro-Ser-Thr), CS-1 fragment (Asp-Glu-Leu-Pro-Gln-Leu-Val-Thr-Leu-Pro-His-Pro-Asn-Leu-His-Gly-Pro-Glu-Ile-Leu-Asp-Val-Pro-Ser-Thr), REDV peptide (Arg-Glu-Asp-Val), CS-5 fragment peptide (Gly-Glu-Glu-Ile-Gln-Ile-Gly-His-Ile-Pro-Arg-Glu-Asp-Val-Asp-Tyr-His-Leu-Tyr-Pro) and the like. Other exemplary binding biofunctional moieties include peptides, such as those described above, or CGLIIQKNEC (CLT1) and CNAGESSKNC (CLT 2) for binding to clots. Various peptides are well known in the art for binding to cells in the brain, kidneys, lungs, skin, pancreas, intestine, uterus, adrenal gland, and prostate, including those described in Pasqualini et al, “Searching for a molecular address in the brain,” Mol. Psychiatry. 1(6) (1996) pp. 421-2 and Rajotte, et al., “Molecular heterogeneity of the vascular endothelium revealed by in vivo phage display,” J Clin. Invest. 102(2) (1998) pp. 430-437, incorporated by reference herein. Other cell adhesion peptides, growth factors, antibodies, sugars, nucleotides, DNA, and the like known in the tissue and bioengineering arts may also be suitable moieties or ligands for the surface(s) of respective phases of the microfibers. Proteins, such as heat shock protein HSP70 for dendritic cells and folic acid to target cancer cells can be suitable ligand moieties for the surface of a phase of a microfiber. Other suitable surface moieties include polysaccharides or sugars, such as silyilic acid for targeting leucocytes, targeting toxins such as saporin, antibodies, including CD 2, CD 3, CD 28, T-cells, and other suitable antibodies are listed in a Table at http://www.researchd.com/rdicdabs/cdindex.htm (Jun. 14, 2007), incorporated by reference. Other suitable binding moieties include aptamers, which are small oligonucleotides that specifically bind to certain target molecules, for example, Aptamer O-7 which binds to osteoblasts; Aptamer A-10 which binds to prostate cancer cells; and Aptamer TTA1, which binds to breast cancer cells. Other binding biological binding moieties suitable for tissue engineering or cell cultures known or to be developed in the art are contemplated by the present disclosure. As noted above, such biofunctional agents are optionally included throughout one or more phases of the multiphasic microfibers or may be provided only on the surface of an exposed phase (as a surface bound moiety), as will be described in greater detail below.

Further, the multiphasic microfibers may include immunotherapeutic agents, such as antibodies and immunomodulators, which may inhibit growth of certain target cells, which include by way of non-limiting example, HERCEPTIN™ (trastuzumab, humanized IgG1 antibody for metastatic breast cancer); RITUXAN™ (Rituximab, chimeric IgG1 antibody for NHL); PANOREX™ (17-1A monoclonal antibody), BEC2 (anti-idiotypic antibody), IMC-C225 (monoclonal antibody); VITAXIN™ (monoclonal antibody); CAMPATHI/H™ (DNA-derived humanized monoclonal antibody), 5G1.1 (humanized IgG for treatment of rheumatoid arthritis (RA), systemic lupus erythematosus (SLE), nephritis); 5G1.1-SC (humanized ScFv antibody for cardiopulmonary bypass, infarction, angioplasty and other cardiac procedures); ABX-CBL (humanized antibody for graft-versus-host disease (GvHD)); ABX-CBL (murine CD147 antibody for allograft rejection); ABX-IL8 (humanized IL-8 antibody for psoriasis); AD-159 (humanized antibody for human immunodeficiency virus (HIV)); AD-439 (humanized antibody for HIV); ANTEGREN™ (humanized IgG antibody for multiple sclerosis); Anti-CD11a (humanized IgG1 antibody for psoriasis); Anti-CD18 (humanized Fab′2 antibody for myocardial infarction); Anti-LFA1 (murine Fab′2 antibody for allograft rejection); Anti-VEGF (humanized IgG1 antibody for cancer); ANTOVA™ (humanized IgG antibody allograft rejection); BEC2 (murine IgG antibody for lung cancer); BIRR-1 (murine IgG2a antibody for stroke); BTI-322 (Rat IgG antibody GvHD); C225 (chimeric IgG antibody for head and neck cancers); CAT-152 (humanized antibody glaucoma); CDP571 (humanized IgG4 antibody for Crohn's disease); CDP850 (humanized antibody for psoriasis); CORSEVIN M™ (chimeric antibody as an anticoagulant); D2E7 (humanized antibody for RA); Hu23F2G (humanized IgG antibody for stroke and MS); ICM3 (humanized antibody for Psoriasis); IDEC-114™ (primatized antibody for psoriasis); IDEC-131™ (humanized antibody for SLE, multiple sclerosis (MS)); IDEC-151™ (primatized IgG1 for RA); IDEC-152™ (primatized antibody for asthma and allergic reactions); INFLIXIMAB™ (chimeric IgG1 antibody for RA, Crohn's disease); LDP-01 (humanized IgG antibody for stroke, allograft rejection); LDP-02 (humanized antibody for ulcerative colitis); LDP-03/CAMPTATH 1H™ (humanized IgG1 antibody for chronic lymphocytic leukemia (CLL)); Lym-1 (chimeric antibody for non-Hodgkin's lymphoma (NHL)); LYMPOCIDE™ (humanized antibody for NHL); MAK-195F (murine Fab′2 antibody for toxic shock); MDX-33 (human antibody for autoimmune haematogical disorders); MDX-CD4 (human IgG antibody for RA); MEDI-500 (murine IgM antibody for treating GvHD); MEDI-507 (humanized antibody for psoriasis and GvHD); OKT4A (humanized IgG antibody for allograft rejection); ORTHOCLONE™ (humanized IgG antibody for autoimmune disease); ORTHOCLONE™/anti-CD3 (murine mIgG2a antibody for allograft rejection); OSTAVIR™ (human antibody for Hepatitis B); OVAREX™ (murine antibody for ovarian cancer); PANOREX 17-1A™ (murine IgG2a antibody for colorectal cancer); PRO542 (humanized antibody for HIV); PROTOVIR™ (humanized IgG1 antibody for cytomegalovirus infection (CMV)); REPPRO/ABCIXIMAB™ (chimeric Fab antibody for complications from coronary angioplasty); rhuMab-E25 (humanized IgG1 antibody for asthma and allergies); SB-240563 (humanized antibody for asthma and allergies); SB-240683 (humanized antibody for asthma and allergies; SCH55700 (humanized antibody for asthma and allergies); SIMULECT™ (chimeric IgG1 antibody for allograft rejection); SMART a-CD3™ (humanized IgG antibody for autoimmune disease, allograft rejection, and psoriasis); SMART M195™ (humanized IgG antibody for Acute Myeloid Leukemia (AML)); SMART I D10™ (antibody for NHL); SYNAGIS™ (humanized IgG1 antibody for RSV); VITAXIN™ (humanized antibody for Sarcoma); and ZENAPAX™ (humanized IgG1 antibody for allograft rejection), and combinations thereof.

In certain aspects, a multiphasic microfiber may comprise biofunctional active ingredient immunotherapeutic agents selected from the group consisting of: Smart M195™, LYMPHOCIDE™, Smart I D10™, ONCOLYM™, rituximab, gemtuzumab, trastuzumab, Anti-LFA1; ANTOVA™; ABX-CBL; ABX-CBL; BTI-322, CORSEVIN M™, IDEC-152; LDP-01; MAK-195F; MEDI-507; OKT4A ORTHOCLONE™/anti-CD3; REPPRO/ABCIXIMAB™; SIMULECT™; SMART a-CD3™; ZENAPAX™, and combinations thereof.

In certain other embodiments, the multiphasic microfibers may further comprise a hormonal treatment agent, such as hormonal agonists, hormonal antagonists (e.g., flutamide, tamoxifen, leuprolide acetate (LUPRON™), LH-RH antagonists), inhibitors of hormone biosynthesis and processing, steroids (e.g., dexamethasone, retinoids, betamethasone, cortisol, cortisone, prednisone, dehydrotestosterone, glucocorticoids, mineralocorticoids, estrogen, testosterone, progestins), antigestagens (e.g., mifepristone, onapristone), antiandrogens (e.g., cyproterone acetate), and combinations thereof, by way of non-limiting example.

Thus, in certain aspects, the multiphasic microfibers of the present disclosure optionally comprise one or more biofunctional agents selected from: anti-rejection drugs (such as cyclosporine), anti-inflammatory agents, non-steroidal anti-inflammatory agents (NSAIDs), COX-I and II inhibitors, antioxidants, antimicrobial agents, including antiviral, antifungal, antibiotics and the like, and combinations and equivalents thereof. For example, useful anti-inflammatory agents include steroids, such as glucocorticoids, betamethasone, dexamethasone, prednisolone, corticosterone, budesonide, estrogen, sulfasalazine, and mesalamine, while indomethacin, ibuprofen, naproxen, and the like are suitable NSAIDs for incorporation into one or more phases of the multiphasic microfibers. Suitable antibiotic agents, include penicillin, cefoxitin, oxacillin, tobranycin, rapamycin, by way of non-limiting example.

Other biofunctional agent materials also include non-genetic therapeutic agents, such as: anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid, amlodipine and doxazosin; antineoplastic/antiproliferative/anti-miotic agents such as paclitaxel, 5-fluorouracil, cisplatin, vinblastine, cladribine, vincristine, epothilones, methotrexate, azathioprine, adriamycin and mutamycin; endostatin, angiostatin and thymidine kinase inhibitors, Taxol™ and its analogs or derivatives; anesthetic or pain-killing agents such as lidocaine, bupivacaine, and ropivacaine; anti-coagulants such as D-Phe-Pro-Arg chloromethyl keton, RGD peptide-containing compound, heparin, anti-thrombin compounds, anti-thrombin antibodies, platelet receptor antagonists, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick anti-platelet peptides; DNA de-methylating drugs such as 5-azacytidine, (also categorized as a RNA or DNA metabolite that inhibits cell growth and induce apoptosis in certain cancer cells); cholesterol-lowering agents; vaso-dilating agents; and agents that interfere with endogenous vasoactive mechanisms; anti-oxidants, such as probucol; angiogenic substances, such as acidic and basic fibroblast growth factors, estrogen including estradiol (E2), estriol (E3) and 17-Beta Estradiol; drugs for heart failure, such as digoxin, beta-blockers, angiotensin-converting enzyme (ACE) inhibitors including captopril, enalopril, and statins and related compounds.

In some aspects, it may be desirable to avoid detection of the multiphasic microfibers by the animal's immune system, for example, to prevent removal or an immune system rejection response from the organism, like a human body, by macrophages and the like. The present disclosure contemplates various methods to prevent an animal's immune system from identifying the microfibers and mounting an immune system response. In addition to the immunomodulator agents discussed above, another method to avoid immune response can be to provide moieties on the surface of at least one phase that is a “cloaking agent,” which prevents the animal's immune system from recognizing a foreign body. Examples of such moieties include modified carbohydrates, such as sialic acid, dextran, pullulan, or glycolipids, hyalluronic acid, chitosan, polyethylene glycols, and combinations thereof. Other examples of immune system cloaking agents known in the art or to be discovered are further contemplated.

Certain suitable biofunctional active ingredients (e.g., pharmaceutically active ingredients or drugs, known to those of skill in the art) include, but are not limited to, low-molecular weight molecules, quantum dots, natural and artificial macromolecules, such as proteins, sugars, peptides, DNA, RNA, and the like, natural polymers, dyes and colorants, inorganic ingredients including nano-materials, and nano-crystals, fragrances, and mixtures thereof.

A variety of low molecular weight molecules can be included in one or more phases of the multiphasic microfibers, particularly those having a molecular weight of less than about 10,000, optionally less than about 1,000, and optionally less than about 500. Such molecules include biofunctional therapeutic drugs, which by way of non-limiting example, including chemotherapeutic drugs, for example, doxorubicin (molecular mass of about 543.5 g/mol); paclitaxel or Taxol™ (molecular mass of about 853.9 g/mol), cholesterol lowering drug, lovastatin (molecular mass of about 404.5 g/mol), NSAID analgesic ibuprofen (molecular mass of 206.3 g/mol). Quantum dots are optically active nano-structures, for example, cadmium tellurium (CdTe).

Macromolecules include a wide range of compounds, generally including polymers and biomolecules having relatively large molecular weights. Such macromolecules can be naturally occurring or synthesized. Amino acids, peptides (amino acids linked via peptide bonds); polypeptides (linear chains of peptides); and proteins (primary, secondary, and tertiary folded polypeptides) are all contemplated as active ingredients. Exemplary active ingredient proteins include heat shock protein 70 (HSP70) for dendritic cells and folic acid for cancer cells. Exemplary toxins for use as active ingredients include saporin and Botulinum toxins. Exemplary sugars include silyilic acid leucocytes and glucuronic acid, for example. Useful nano-components and nano-crystals generally having a particle size of less than about 50 nm, optionally less than about 20 nm, and in some aspects, less than 10 nm. Useful non-limiting active ingredient nanoparticles include magnesium oxide, and metal based nano-particles, comprising gold, silver, and the like. Suitable active ingredient nano-crystals include magnetite (Fe₃O₄).

In certain embodiments, each phase can comprise a different biofunctional compound throughout the phase, can comprise a surface moiety (e.g., each phase's surface can be tagged with a different targeting moiety or active agent) or each phase can optionally have different surface properties. Specifically, at least one phase can be selected to be hydrophilic, hydrophobic, positively charged (cationic), negatively charged (anionic), surface active agent modified (e.g., PEG-ylated or covered with a zwitterion), superhydrophobic, superhydrophilic, olephobic, olephilic, and/or nanostructured, as described above. A multiphasic microfiber can be designed to have such properties by providing such materials within the material forming the phase, or may be provided by subsequent treating, reacting, or coating of the exposed phase surface after formation of the multiphasic microfiber to achieve such properties. Polymers within a selected phase can further be modified to interact and/or react with certain target moieties. For example, reactive groups on a polymer in a first phase may be cationic and the desired moiety for the surface is anionic and will be attracted to the surface of the first phase.

In other embodiments, the functional groups on the polymer may participate in a reaction with a functional group present on a given moiety, such that they react and are bonded to the surface of the phase. For example, if a first phase of the multiphasic microfiber has a polymer with a —CHO functional group at the surface and the surface moiety to be attached to the first phase has a —CH₂NH₂ functional group, such groups have an affinity to form a —C═N covalent bond, thus, the surface of the first phase has an affixed moiety presented at the surface. In certain aspects, a CVD coating may be applied to a surface of a phase which has functional groups that are chemical reactive toward the coating to form a covalent bond. For example, the —CHO and —CH₂NH₂ groups have an affinity to form a rigid —C═N bond and a resulting adhesion layer. Cell adhesion ligands, such as a laminin-derived IKVAV-containing peptide sequence, can be covalently bonded to only one phase/compartment having reactive functional groups applied thereto. By way of non-limiting example, when multiphasic fiber scaffolds are modified via spatially controlled peptide immobilization for “surface patterning,” a unique type of scaffold is obtained, which exhibits highly selective cell guidance (≦0.05) at spatial pattern resolutions (<10 μm) that have only previously been observed for flat substrates.

In other variations, additional ingredients that can be used in the multiphasic microfibers are not biofunctional, but rather are used for diagnostic purposes, such as in various diagnostic medical imaging procedures (for example, radiographic imaging (x-ray), fluorescence spectroscopy, Forster/fluorescent resonance energy-transfer (FRET), computed tomography (CT scan), magnetic resonance imaging (MRI), positron emission tomography (PET), other nuclear imaging, and the like). Diagnostic ingredients for use with diagnostic imaging include contrast agents, such as barium sulfate for use with MRI, for example, or fluorescein isothiocyanate (FITC).

Other conventional biocompatible materials can be used to form the materials of respective phases, including solvents, plasticizers, cross-linking agents, surface active agents, fillers, bulking, or viscosity modifying agents, pH modifiers, pH buffers, antioxidants, impurities, UV stabilizers, and where appropriate, flavoring, or fragrance substances.

By guiding cell growth on selective areas of a microfiber scaffold, other regions or phases of the microfiber can be independently controlled to perform functions, such as active biofunctional compound and/or drug delivery to the surrounding environment. In certain variations, the multiphasic microfibers of the present disclosure are designed to interact with target cells. Target cells can be of human origin (autologous or allogeneic) or from an animal source (xenogeneic), or genetically engineered. Such cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells, for example. For example, selective growth of fibroblasts, or other cell lines such as nerve cells, hepatocytes, epithelial cells, endothelial cells, and the like are contemplated. Furthermore, control over cell proliferation area also presents opportunities for co-culture of two (or more corresponding to the number of phases in the microfiber) cell lines. The multiphasic microfibers of the disclosure advantageously provide truly three-dimensional environments, where different types of cells interact with each other, which among other things, provides the ability to improve understanding of processes such as cell-to-cell signaling.

In various aspects, a multiphasic microfiber has an evident long axis or longitudinal major axis, which may be nominally defined as a “z” axis (see FIG. 10 with axes labeled “a”) and may further have a surface pattern defined by different properties of respective phases. In certain aspects, the axis may be defined relative to the microfiber; i.e., the axis may follow the curvature of the microfiber as it twists around 3-dimensional space. As such, multiphasic microfibers have two-dimensional and optionally three-dimensional compositional variation, for example, for example, based on respective patterns optionally formed on the surface. In certain alternate variations, a compositional gradient may be provided within a single phase to correspond to the same or distinct directions in other phases. For example, a microfiber phase can be designed to have a compositional gradient of a particular composition, biofunctional compound, or surface property having a gradient in a particular direction. Such compositional gradients can be independently selected for each respective phase to generate different patterns. Further, the exposed surfaces of each phase may have distinct geometric patterns formed thereon, which can be designed via masks (e.g., used during surface coating applications) and similar design techniques.

Thus, in various aspects, the present disclosure provides microfibers that provide a substrate with the ability to have two- or three-dimensional micro-geometry bonding to form complex device structures for bioMEMs (biological micro-electromechanical systems) applications that provide flexibility in the active agents to be delivered in vivo or ex vivo. Such multiphasic microfibers are clean, dry, more flexible for a wide range of substrates, and even more robust in bonding strength.

Electrohydrodynamic co-spinning processes can optionally create multiphasic microfiber scaffold sheets by introducing two or more parallel outlet flows to yield microfibers with multiple independent phases. Co-electrospinning from multiple nozzles adjacent to one another results in a facile transition to three and four compartmental fibers with the interface between different solutions being maintained in a superior fashion. The orientation of individual compartments can be manipulated through relative macroscopic configuration of needles during co-electrospinning. A linear arrangement of three needles can create a microfiber with elongated phase compartments positioned side-by-side, whereas a triangular needle configuration gives rise to “pie” shaped anisotropy, where each phase has an exposed surface. In the side-by-side triphasic fibers, switching the arrangement of capillaries containing the red, blue and green dye loaded solutions leads to corresponding changes in the fibers. This process is conveniently extended up to a plurality of phases, from two ranging up to hundreds of phases. In certain variations, the multiphasic microfibers comprise from 2 to 10 distinct phases, optionally from 3 to 10 distinct phases, and in certain embodiments from 4 to 7 distinct phases.

### Example 1

### Biphasic Jetting

The experimental setup for the present experiment conforms to that of FIG. 1. Two jetting liquids (Fluid A and Fluid B) are fed using dual syringe applicator assembly (FibriJet® SA-0100, Micromedics, Inc., MN, USA) as shown in FIG. 1. In this setup, two 1 ml syringes are controlled by one syringe pump. Each syringe is filled with separate jetting solutions. These two syringes are connected to a dual channel tip (Fibrikf™ SA-0105, Micromedics, Inc., MN, USA) which has a dual cannula with a dimension of 26 gauge and 3 inch length. These dual cannula or capillaries are covered with a transparent plastic tube that ties these two capillaries in side-by-side fashion. In order to avoid the capillary pressure caused by the groove between the two round shape cannula and create a stable biphasic pendent droplet from the side-by-side capillary setup, the tip end level is made even by the sharp cutting of the two capillaries and the plastic tube.

In this example, the material properties of Fluid A and Fluid B (both liquids) are similar. Here, compatibility between the two jetting solutions is desirable to achieve a stable interface between the two phases, and basic components (i.e., polymer and solvent) can be the same to achieve similar viscosity, surface tension, and the like. However, each side can include a different active ingredient that is maintained in each phase throughout the process. Preventing diffusion of these different active ingredients between phases (from one phase to the other) is usually avoided until the point of solidification. In line with the above mentioned objectives, mixtures of a biofunctional agent suspended in water and mixed with polymer for each side of the jetting solution.

Fluid A is a solution composed of a first biocompatible polymer and a biofunctional agent. Fluid B is a second biocompatible polymer combined with the same biofunctional agent. These fluids are jetted in an electrohydrodynamic jetting apparatus, where 8 kV of electric potential is applied between 25 cm separation of the electrodes. A glass slide is covered with aluminum foil except about 80% of the surface of one face, and the jetting is performed on the open face of the glass slide. Electrodes are connected directly to the side-by-side capillaries and the aluminum foil covering the glass slide substrate. The same flow rate is set for each side.

### Example 2

### Multiphasic Microfibers Having Charged Surfaces

An aqueous solution of 95 weight % polyethylene imine (PEI), and 5 wt. % poly(acryl amide-co-acrylic acid) (PAAm-co-AA) is co-jetted with an aqueous solution of 95 wt. % poly(acrylic acid) (PAA) and 5 wt. % PEI. Each respective solution is loaded with a different colorant (dye), for example, one colorant may be selected to be dextran and another fluorescein isothiocyanate (FITC). Two parallel polymer flows are introduced in a nozzle that contains inlets in a side-by-side geometry, such as is shown in FIG. 1, described above. Under these conditions, a droplet forms at the tip of the nozzle. Upon application of a sufficiently strong electrical field (about 5 to about 10 kW) between the nozzle and a counterelectrode, which serves as the collector, a polymer thread is ejected from the droplet resulting in biphasic microfibers, where one phase is predominately positively charged and the other phase is predominately negatively charged.

### Example 3

Poly(lactide-co-glycolide) with a lactide:glycolide ratio 85:15 (M_{w} 50-75,000 g/mol), poly[(mphenylenevinylene)-alt-(2,5-dibutoxy-p-phenylenevinylene)] (MEHPPV), poly[tris(2,5-bis(hexyloxy)-1,4-henylenevinylene)-alt-(1,3-phenylenevinylene)] (PTDPV), fluorescein isothiocyanate (FITC), chloroform, and N,N′-dimethyl formamide (DMF), copper sulfate pentahydrate (CuSO₄.5H₂O), sodium ascorbate, phosphate buffered saline (PBS), and Tween-20 are used, which are commercially available from Sigma-Aldrich, USA. Polythiophene dyes, ADS 406PT and ADS 306PT are purchased from American Dye Source, USA.

The basic experimental setup used for fabrication of fibers includes two syringes containing the polymer solutions held together using a syringe holder, as described in Examples 1 or 2, above. Each of the syringes is connected to a capillary needle (diameter: 26 gauge, length: 8.2 cm, commercially available from Micromedics Inc, USA). The capillaries are connected to the cathode of a DC voltage source (Gamma High Voltage Research, USA). The flow rate is controlled by a syringe pump (Kd Scientific, USA). The syringe/syringe pump assembly is placed on top of a rectangular box frame with aluminum support and a Plexiglas top. Microfiber scaffolds are deposited onto an aluminum foil covered spinning wheel (Synthecon, Inc., modified to experimental requirements) rotating at 20 rpm and placed at a distance of ˜5 cm from the capillary tip. The experiments are carried out at room temperature inside a fume hood with an average face velocity of 0.1 m/s.

Three liquid streams are co-jetted, where each liquid stream has 18 w/w % of 85:15 PLGA in 95:5 chloroform:DMF solvent. The three streams are co-jetted at 0.03 mL/h in an electric field with an applied voltage of 9 kV. Respective phases contain blue fluorescence (B) (poly[(m-phenylenevinylene)-alt-(2,5-dibutoxy-p-phenylenevinylene)] (MEH-PPV) colorant), green fluorescence (G) (poly[tris(2,5-bis(hexyloxy)-1,4-phenylenevinylene)-alt-(1,3-phenylenevinylene)] (DPV) colorant), and red (R) fluorescence (substituted polythiophene (ADS306PT) colorant). Each of these colorants is commercially available from Sigma-Aldrich. Three, four and seven phasic fibers are prepared by employing a corresponding number of capillaries in the desired spatial arrangement.

### Example 4

### Fiber Analysis

Scanning Electron Microscopy (SEM): The microfiber scaffolds are spun on top of the aluminum substrate, sputter-coated with gold and their surface morphology is examined by a Scanning Electron Microscope (Philips XL30 ESEM, high vacuum mode). Confocal Laser Scanning Microscopy (CLSM): CLSM micrographs are obtained with a FluoView 500 confocal laser scanning microscope (Olympus, Japan). MEHPPV (and ADS406PT), PTDPV, and ADS306PT are excited by 405 nm UV, 488 nm Argon, and 533 nm Helium-Neon green lasers respectively. Optical filters of emission wavelength 430-460 nm, 505-525 nm, and 560-600 nm are used to visualize the fluorescence of MEHPPV, PTDPV, and ADS306PT respectively. For cross-sectional analysis, microfiber scaffolds are sectioned perpendicular to fiber length using a cryostat microtome (HM5500MC, Microme, USA) maintained at −18° C. The sections are collected on a glass slide (Fisher Scientific, USA) and imaged via CLSM.

Electrohydrodynamic co-spinning of two or more solutions according to the present disclosure yields multiphasic microfiber scaffolds with individually functionalizable phases or compartments. Schematic representation of the process for preparation of biphasic PLGA microfibers using a dual capillary assembly (red and blue), was described above in FIG. 1. This approach can be extended to fabrication of multiphasic microfibers by incorporating additional outlet streams (not shown), as discussed above.

In FIG. 5B, an SEM image of biphasic microfiber scaffold and FIG. 4A, a CLSM image of biphasic microfiber scaffold shows both a distinct biphasic geometry and excellent alignment of fiber phases. Acetylene-PLGA can be selectively added to one of the compartments to provide functional groups for subsequent micro-structuring, as described above and further herein.

FIGS. 6A through 6M show various multiphasic nano-components formed in accordance with the methods of the present disclosure with biodegradable PLGA polymers, including aligned multiphasic microfibers. FIGS. 6A and 6C-6D show SEM and CLSM, respectively, aligned biphasic fibers (where 6D is the cross-sectional view).

FIGS. 6E through 6K show aligned triphasic microfibers and their respective cross-sections, as described above, where three side-by-side capillaries are used to electro-hydrodynamically co-jet three phases respectively containing red, green, and blue in left, central and right orientation of syringes (FIG. 6D is RBG orientation; FIG. 6E is RGB orientation; and FIG. 6F is BRG orientation) to provide different triphasic repeating patterns of aligned fibers.

FIG. 6B is an SEM of a microscopic ordered bundle of fibers prepared in accordance with the present teachings. Further, CLSM images of tetraphasic fibers having different phase orientation are shown in FIGS. 6L-6M, formed by jetting four distinct phases. FIG. 6L shows alternating phases 1-4 (ABCD pattern), as where FIG. 6M is formed via a diamond pattern of jetting the respective four phases, namely a diamond pattern is formed by phases 2 and 3 adjacent phases 1 and 4.

### Example 5

In another example, an 18:100 w/w ratio of PLGA in a 95:5 (v/v) mixture of chloroform/DMF is pumped through each of the needles at a flow rate of 0.02 ml/h. One of the solutions is loaded with ADS306PT (red dye), whereas the other solution contains MEHPPV (a green dye). Application of DC potential (8.1-8.3 kV) results in droplet stretching, cone formation, and ejection of a solid fiber, which deposits on the counter electrode. This process is extremely stable, does not show any bending or whipping instabilities and can be carried on uninterrupted for at least about 5 hours.

Biphasic droplets formed with such solutions have a distinct interface between the two jetting solutions, which is maintained throughout the jetting process. FIG. 5A is an SEM micrograph of a highly aligned fiber scaffold sheet formed by electrohydrodynamic spinning onto a wheel assembly rotated at 16-18 revolutions per minute (rpm). The sheet has a length spanning 3 cm equal to the diameter of the wheel.

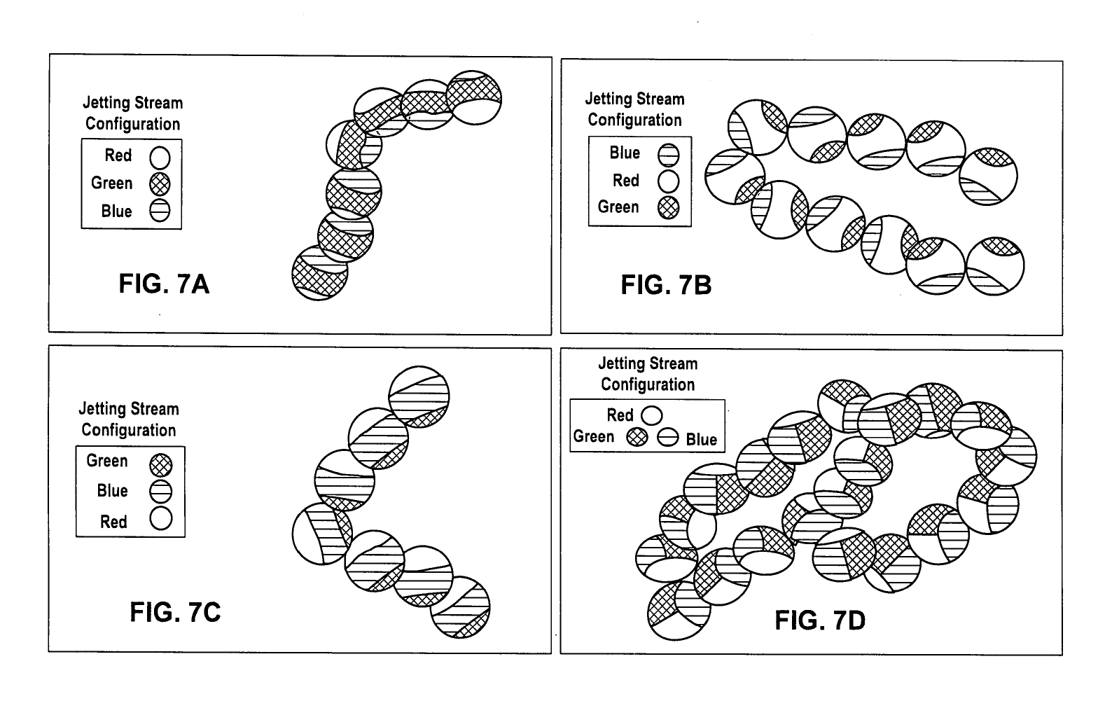

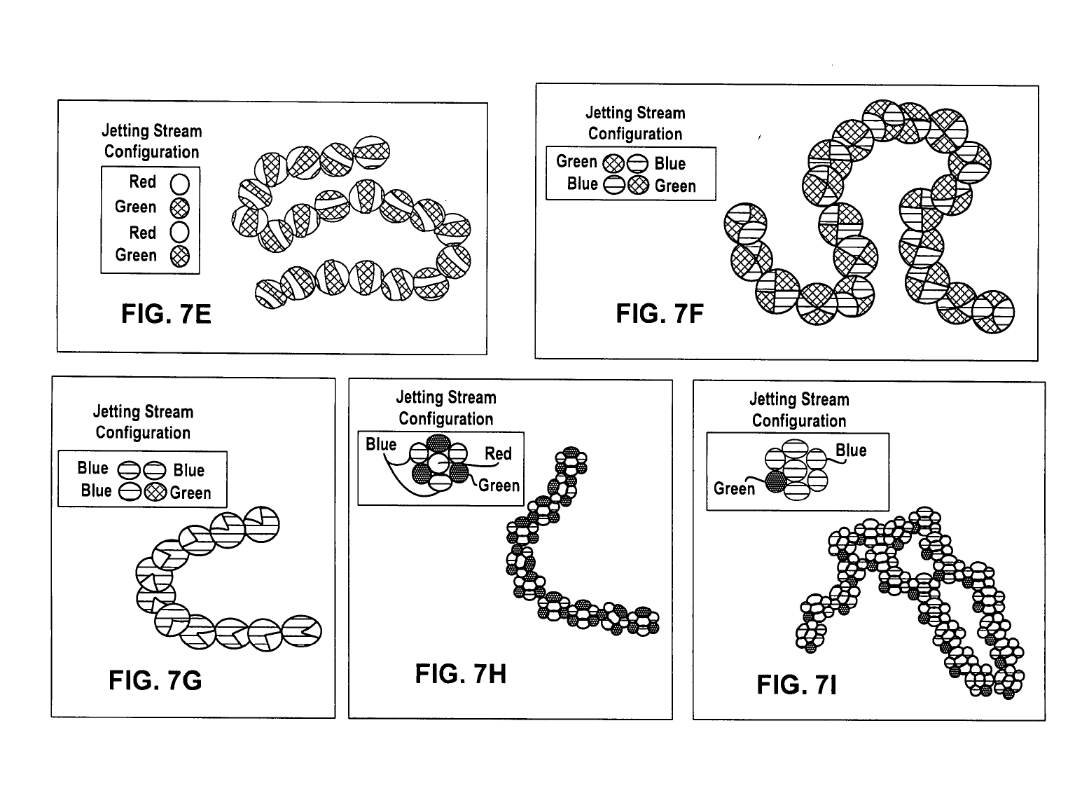

The use of electrohydrodynamic co-spinning processes of the present disclosure further creates scaffolds with a higher number of distinct phases, for example, in excess of seven or more outlet streams can be used simultaneously. In FIGS. 7A-7I, electrohydrodynamic co-spinning of multiphasic microfibers with three to seven distinct phases are shown. When the jetting solutions are arranged in alternating sequence, scaffolds are prepared, which include striped microfibers with 4 distinguishable phases in series (e.g., FIG. 7E). The precision of a plurality of phases in the microfibers and the excellent alignment of such phases is comparable to the alignment and spatial design discussed above for bi- and tri-phasic fibers. To fabricate tissue scaffolds with more than two independent phases in each fiber, a ternary nozzle configuration setup is used. If a square arrangement of outlet flows is used, the phases of the microfibers are arranged as rosettes of alternating phases (FIG. 7F). Similarly, more complicated rosettes consisting of seven phases are prepared (FIG. 7H), which have a striking resemblance to a flower shape.

All images in FIGS. 7A-7I schematically represent overlays of cross sectional views of fluorescence-generated images. Blue (B), green (G) and red (R) fluorescence represent MEHPPV, PTDPV, and ADS306PT dyes, respectively. Insets indicate number, spatial configuration, and nature of outlet streams used during electrohydrodynamic co-jetting, such as shown in FIG. 1, but with up to 7 separate syringes in the configuration. FIGS. 7A-7C show triphasic microfiber isomers prepared using a nozzle with sequential arrangement of inlet flows FIG. 7A: {sRGB}; FIG. 7B: {sBRG}; and FIG. 7C: {sRBG}.

In contrast to the striped isomers (FIGS. 7A-7C), a triangular arrangement of the outlet flows result in very distinctive {pRGB}-type fiber architecture (FIG. 7D). In FIG. 7D, a multiphasic microfiber scaffold is prepared using a triangular nozzle configuration resulting in a “pie”-shaped anisotropy, designated by a “p” sequence: {pRBG}. Longitudinal Z-stack analysis of the {pRGB}-isomer confirms the unique microstructure.

In FIG. 7E, a tetraphasic microfiber scaffold showing alternating red and green phases is prepared using a sequential configuration of four outlet streams. FIG. 7F, a tetraphasic microfiber scaffold resulting from a tetragonal arrangement of outlet streams, where opposite streams carry the same dye (B and G). In FIG. 7G, a tetraphasic microfiber scaffold with one out of four phases of each fiber labeled with PTDPV, yielding a green “quarter” phase, and a three-fold larger blue phase. FIG. 7H, shows a heptaphasic microfiber scaffold resembling a flower shape.

The ability to precisely control internal fiber architectures includes the ability not only to control phase arrangement, but also the relative size of phases within the microfibers. FIGS. 7G and 7I show biphasic microfiber scaffolds with asymmetric phase sizes. In these examples, one phase is three- or six-times larger than the other phase. For example, in FIG. 7I, heptaphasic fibers are formed where one green phase is six-fold times smaller than the other phases. Using electrohydrodynamic co-jetting, the synthesis of these microfibers can be simply achieved by charging an appropriate number of outlet flows with identical jetting solutions. In this way, the phases can be successively increased and relative phase sizes can be controlled with high accuracy.

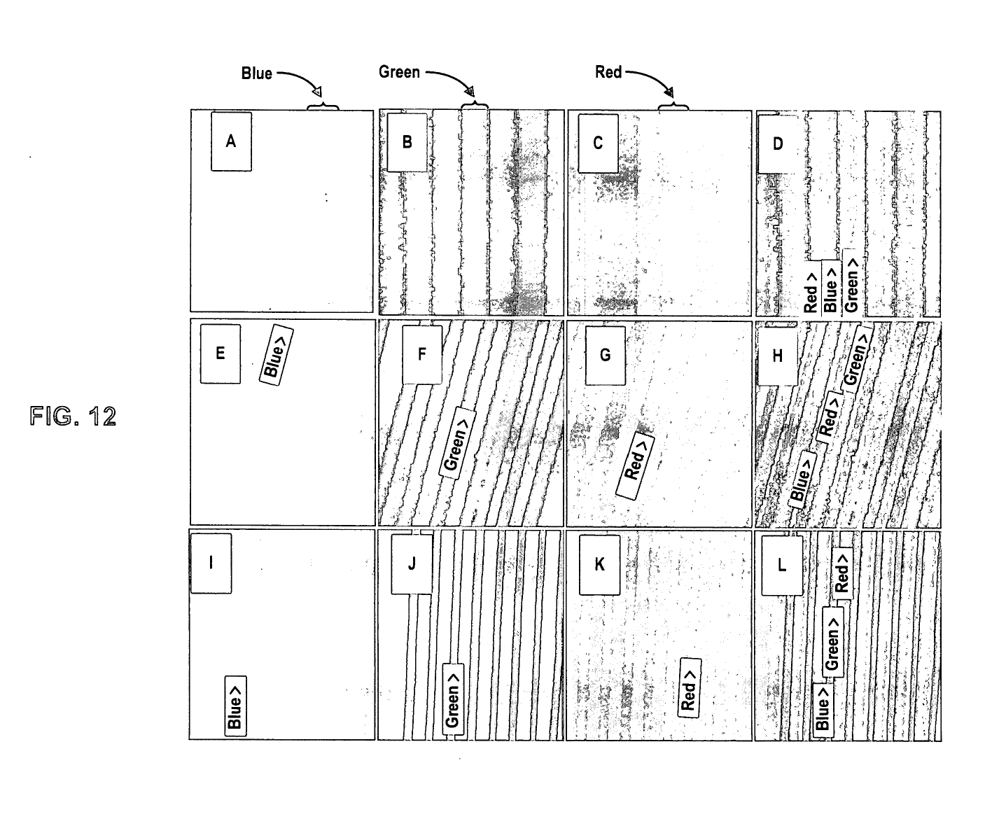

In FIGS. 12A-L, triphasic microfiber scaffolds are made by electrohydrodynamic cospinning techniques described above. FIGS. 12A-L show CLSM micrographs of triphasic microfiber scaffolds prepared by side by-side co-jetting of three different PLGA solutions obtained at 405, 488, and 533 nm excitation wavelengths having three individual and distinct phases. All images depict top views. Individual blue (B), green (G), and red (R) micrographs representing fluorescence from MEHPPV, PTDPV and ADS306PT dyes are shown independently (blue images in FIGS. 12A, 12E, and 12I, green images in FIGS. 12B, 12F, and 12J, and red images in FIGS. 12C, 12G, and 12K) followed by a composite or overlay of the three individual fluorescence images (from left to right—FIGS. 12D, 12H, and 12L, respectively). Different sequences of outlet streams used during electrohydrodynamic co-jetting give rise to different fiber isomers. FIGS. 12A-D: {sRGB}; FIGS. 12E-H:{sBRG}; and FIGS. 12I-L: {sRBG}; where “s” stands for “striped” or “sequential” and the letters denote the fluorescent labeling of solutions in the order, in which they are positioned during co-spinning.

For simplicity, each inlet stream comprises the same base polymer, PLGA, dissolved in a mixture of chloroform and N,N′-dimethyl formamide, and is blended with appropriate polymer additives as needed for subsequent imaging. FIGS. 12A-L micrographs reveal that dyes initially loaded in the different jetting solutions remain isolated in the corresponding phases after solidification. In addition, extremely sharp boundaries are observed at the interfaces between individual phases suggesting minimal mass transfer between phases. In spite of the added complexity that is associated with the transition from two to three outlet streams, the new fibers show the same, high-precision alignment of fibers and phases, already observed for biphasic microfiber scaffolds (e.g., FIGS. 4A and 5B).

For embodiments where the multiphasic micro-structured fibers have more than two phases, the configuration of individual phases becomes another design parameter. For a fiber with three equally sized phases observed in a three dimensional space, there are in theory optically distinguishable permutations. In the following examples, all four isomers theoretically possible for a triphasic fiber are created by the electrohydrodynamic cospinning methods of the present disclosure, demonstrating versatility of the present methods and microfibers. The corresponding fiber isomers comprise fibers with three striped phases, i.e., red, blue, green {sRBG}, blue, red, green {sBRG}, and blue, green, red {sBGR} phases. In addition, one trigonal isomer exists, which we will refer to as “pie-shaped” fiber {pRGB}. Due to the stable jet and well-defined interface during biphasic co-spinning, changing the macroscopic arrangement of outlet streams results in different fiber isomers. For this purpose, a set of three nozzles is employed for electrohydrodynamic co-spinning, where the sequence of the incoming jetting solutions as well as their relative arrangement is controlled. Otherwise, the setup of the electrohydrodynamic co-jetting conditions is relatively unaltered from conditions to form biphasic microfibers. FIGS. 12A to 12C show triphasic PLGA fibers, where the three outlet streams of the nozzle are placed in sequence. If the jetting solution containing the green dye is placed in the center, the {sBGR}-isomer is prepared (FIGS. 12A-D). Placing the red or blue jetting solutions into the center space results in {sBRG}- or {sRBG}-isomers, respectively (FIGS. 12E-H and 12I-L). Corresponding confocal images confirm equal sizes of the individual phases, their precise alignment, and the sharp boundaries between individual phases.

The electrohydrodynamic co-jetting process is well-suited to not only control the exact topology of the deposited fiber scaffolds, but also their phase architecture and presentation with superior precision.

### Example 6

### Selective Surface Modification of Multiphasic Microfibers with Biofunctional Moieties

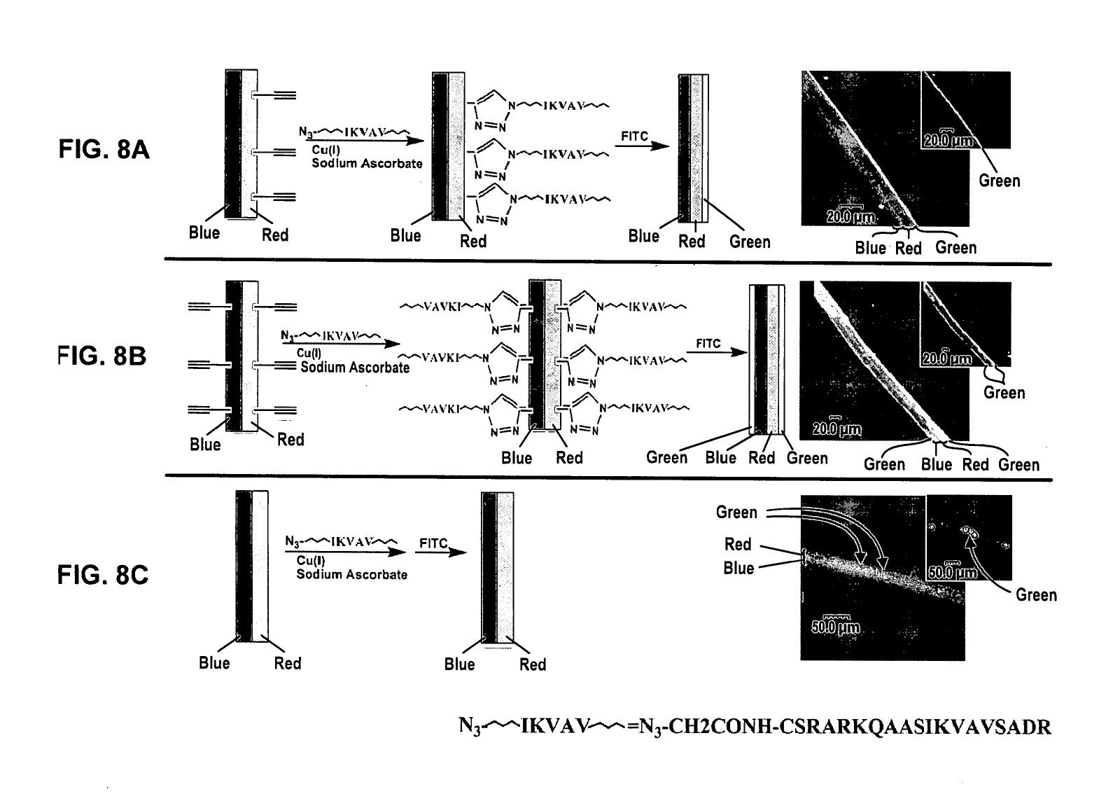

Poly(lactide-co-propargyl glycolide) (acetylene-PLGA) is added to one of the jetting solutions and a biphasic fiber scaffold is prepared via electrohydrodynamic co-spinning, with a set-up as described in Example 1, above. The resulting scaffolds are reacted via copper-catalyzed Huisgen heterocycloaddition with an azide-modified derivative of the laminin-derived peptide, which includes the functional IKVAV sequence (FIGS. 8A and 8B). The laminin-derived peptide is N₃—CH₂CONH—CSRARKQAASIKVAVSADR, M_{w}=2117 g/mol (peptide-azide) commercially available from TianMa pharmaceuticals, China. The microfiber scaffold (length ˜2 cm) is fixed on the aluminum substrate using tape, and incubated with 150 μL of a 0.47 mM peptide-azide solution in DI water, 50 μL of 0.01 M aqueous CuSO₄.5H₂O solution, followed by 50 μL of 1M aqueous sodium ascorbate solution. The reaction is carried out in 2 ml of DI water containing 0.01% v/v Tween-20 (Sigma, USA) for 10 hours. The unreacted peptide is removed by washing with a 1% v/v Tween-20 in PBS. The fibers are re-suspended in DI water and incubated with 10 μL of 0.01M FITC (dissolved in DMF) for 5 hours. The un-reacted FITC is removed by repeated washing with 1% v/v Tween-20 in PBS.

FIGS. 8A-8C show selective peptide modification of biphasic microfibers demonstrating superb spatial resolution. As noted above, an exposed phase of the microfiber is selectively surface modified with an azide-functionalized cell-binding laminin derived IKVAV peptide (N₃—CH₂CONH—CSRARKQAASIKVAVSADR). Individual CLSM micrographs show blue, green and red fluorescence overlays with inlays representing green fluorescence due to reaction of the peptides with a FITC-probe.

Verification that the laminin peptide is indeed immobilized to only one of the phases present in the multiphasic microfibers is conducted by labeling primary amino groups of the immobilized peptides by reaction with a fluorescence probe (FITC) and subsequently analyzed by CLSM (FIGS. 8A-C). Green fluorescence arising from the FITC probe is only observed for the red phase, i.e., the phase, which contains acetylene-PLGA (FIG. 8A). In FIG. 8A, a biphasic fiber containing free-acetylene groups in one phase only is shown, prepared by blending PLGA with a poly(lactide-co-propargyl glycolide) (about 30 wt % of polymer). The acetylene-containing phase is further loaded with ADS306PT (red fluorescence marker), and the other compartment is labeled with PTDPV (blue fluorescence marker). The fibers react with an azide-functionalized peptide via copper-catalyzed Huisgen 1,3-dipolar cycloaddition. The free amine groups of lysines in the peptide then react with a green-fluorescent dye (FITC) giving rise to green fluorescence in areas, where the reaction occurs. Uniform peripheral green fluorescence due to FITC is seen alongside the red phase only, indicating advantageous selective surface modification.

In a control experiment with biphasic microfiber scaffolds containing acetylene-PLGA in both phases, the peptide is bound to the red and blue phases as evidenced by the uniform distribution of green fluorescence originating from surface-conjugated FITC (FIG. 8B). In FIG. 8B, a biphasic microfiber containing acetylene-PLGA in both phases is subjected to identical conditions as those that formed FIG. 8A, resulting in green fluorescence alongside both phases.

To exclude the possibility of non-specific binding of the laminin peptide, biphasic microfibers without acetylene-PLGA are subjected to the copper-catalyzed Huisgen heterocycloaddition in the presence of the peptide. FIG. 8C, a microfiber without acetylene groups does not exhibit significant green fluorescence indicating negligible non-specific binding of the peptide. In FIG. 8C, a fiber without acetylene groups in either phase is shown, which exhibits negligible green fluorescence, thus indicating negligible non-specific binding of the peptide. This confirms that covalent immobilization of the laminin peptide is achieved in a spatially controlled fashion.

Thus, in certain aspects, the multiphasic microfiber scaffolds of the present teachings are particularly suitable for cellular contact guidance, because they provide the ability to permit spatially controlled presentation of biological information. In this regard, multiphasic microfibers can be micropatterned via individual phases of a biphasic microfiber scaffold being surface-modified with a biofunctional moiety, such as a laminin-derived cell adhesion peptide.

### Example 7

### Cell Incubation Assay

In this example, biphasic PLGA microfiber scaffolds are modified selectively with the azide-functionalized laminin peptide N₃—CH₂CONH—CSRARKQAASIKVAVSADR, which contains an IKVAV active sequence, as described in Example 6. Samples are incubated with NIH 3T3 fibroblasts (sold by ATCC, USA) cultured in T75 culture flasks in Dulbecco's Modified Eagle Medium (DMEM commercially available from Invitrogen, USA) with 10% fetal calf serum (FCS) under 37° C./5% CO₂ conditions. The cells are passaged at recommended confluence. Passages 5-9 are used for all experiments. Glass slides (Fisher Scientific) are modified with polyethylene glycol according to previously reported protocols. See for example, Papra, et al., “Characterization of ultrathin poly(ethylene glycol) monolayers on silicon substrates,” Langmuir 17, 1457-1460 (2001), incorporated by reference herein.

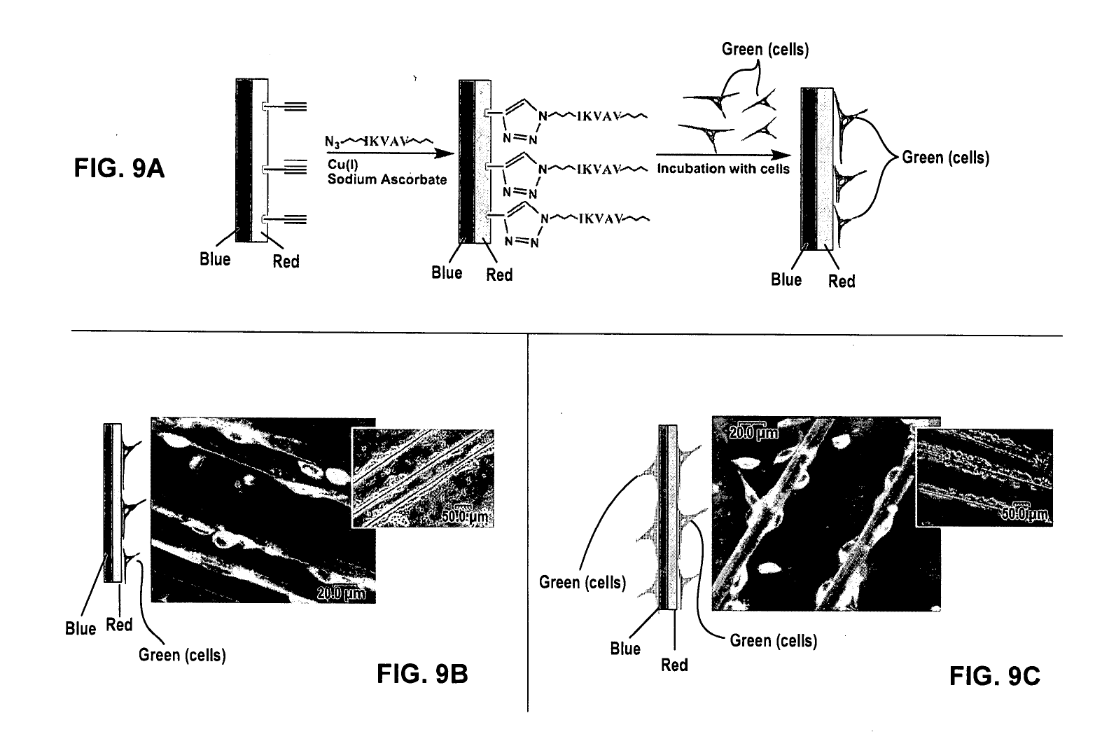

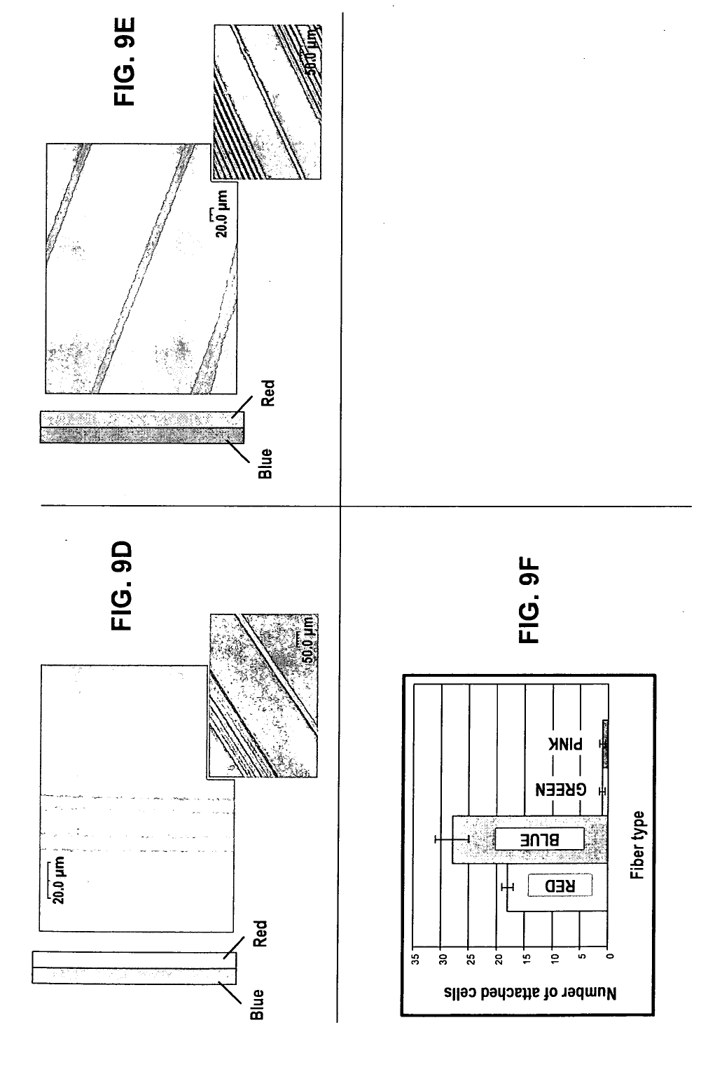

With selectively modified multiphasic microfibers, the biological function of micro-structured multiphasic microfiber scaffolds of the present teachings are assessed in cell culture experiments (FIG. 9A). For this purpose, low density microfiber scaffolds are deposited onto glass slides and selectively modified with the laminin peptide. Biphasic microfiber scaffolds are modified by immobilizing the laminin peptide in one or two exposed phases (FIG. 9A). In addition, biphasic microfibers without acetylene-PLGA in either one of the phases, but treated with the laminin peptide under copper-catalyzed Huisgen heterocycloaddition chemistry conditions and biphasic microfibers containing acetylene-PLGA in both phases, but without peptide modification are used as negative controls.

The four sets of samples are incubated with NIH 3T3 fibroblasts multi-well plates and a cell suspension in serum-free media is added to the samples at a concentration of 1×105 cells/cm² and incubated for 6 hours under culture conditions. Quantification of cell adhesion data is performed for 5 experimental trials by counting number of adhered cells on each microfiber type for a standard length of a single microfiber. Statistical analysis is carried out using a two-tailed Students t-test with unequal variance. For confocal imaging, a live-cell actin stain BODIPY-TMR-Cytochalasin D (sold by Invitrogen) is added to the media after 6 hours of incubation. After 4 hours, the samples are imaged using phase contrast microscopy (e.g., CLSM). The microfibers are imaged using phase contrast microscopy and CLSM to observe relevant cell functions, such as cell spreading and cell orientation relative to the microfibers.

In FIGS. 9A-F, the CLSM images are shown along with insets representing the corresponding phase contrast images. Taken together, the images confirm cell adhesion occurs only on the surfaces of phases of microfibers that are modified with laminin peptide (FIGS. 9B and 9C). In the sample group, where only the red phases are modified with the laminin peptide, cells adhere selectively to the red phase only (FIG. 9B). Biphasic microfibers, where both phases have been surface-modified with the laminin peptide, desirably show rather uniform cell adhesion throughout the fiber scaffold (FIG. 9C). Moreover, the CLSM images reveal that extensive cell spreading occurs only on fiber phases that have been modified with the cell adhesion peptide, i.e., the red phases for selectively modified microfibers (FIG. 9B) and the red and blue phases for uniformly modified fibers (FIG. 9C). The cell adhesion and spreading on the microfibers in (FIGS. 9B and 9C) shows that the microfiber exhibited surface peptide ligands at a concentration sufficiently high to be recognized by the fibroblasts. In contrast, negligible cell adhesion is observed on surfaces that are void of the laminin peptide (FIGS. 9D and 9E).

In FIGS. 9A-9F, guided cell adhesion occurs on biphasic microfiber scaffold selectively modified with a laminin peptide. When IKVAV is immobilized on both phases/compartments (FIGS. 9A, 9D, 9H), cells grow along both phases. In FIG. 9A, a schematic shows selective surface conjugation of the azide-peptide on red phases only. The fiber mesh is then used as scaffold for fibroblast adhesion. FIGS. 9B-9E show CLSM images as well as phase contrast micrographs (inserts) of biphasic microfibers. In FIG. 9B, acetylene-PLGA is incorporated in only the red phase followed by selective peptide conjugation resulting in cells adhesion alongside the red phase only. In FIG. 9C, acetylene-PLGA introduced in red and blue phases results in cells adhesion on both phases. FIG. 9D, shows acetylene-PLGA introduced in either phase resulting in negligible cell adhesion. FIG. 9E, acetylene-PLGA is provided in both phases, but not conjugated with peptide resulting in negligible cell adhesion. In FIG. 9F, a plot quantifying cell adhesion for each fiber type is provided with data plotted as an average of 5 experimental sets. Bars 1, 2, 3 and 4 in the plot represent average number of cells (±S.D.) attached to a standard length of fiber type described in FIG. 9B-9E, respectively. *p≦0.05 for data set 1 when compared to set 3 and 4, n=5. *p≦0.05 for data set 2 when compared to 3 and 4, n=5.

FIGS. 9B, 9E, and 9G show optical and CLSM micrographs of NIH 3T3 fibroblasts cultured on biphasic PLGA microfibers selectively immobilized on only one phase/compartment by reacting the azide-functionalized laminin peptide N₃—CH₂CONH—CSRARKQAASIKVAVSADR, which contains an IKVAV active sequence. When IKVAV is immobilized on both phases/compartments (FIGS. 9A, 9D, 9H), cells grow along both phases. A comparison is provided in FIG. 9C, where microfibers containing free acetylene groups in both phases are subjected to similar conditions to those that formed the microfibers in FIG. 8A, but in the absence of IKVAV (FIG. 9C) and microfibers without free acetylene groups incubated with IKVAV under the same conditions as those that formed FIG. 9A (FIG. 9F), neither of which exhibit any cell adhesion whatsoever.

Cell adhesion is quantified for the four groups corresponding to FIGS. 9B to 9E by counting the number of cells per normalized length of fiber on the basis of phase contrast images. Data is plotted as an average of 5 experiments (FIG. 9F). The quantitative analysis indicates that the number of attached cells depends on the peptide-modified surface area of the respective exposed phases surfaces of the microfiber scaffolds. Samples modified with the laminin peptide in either one or two phases are significantly different from the sample groups without peptides (p≦0.05). The average number of cells that attach to a biphasic microfiber with selective surface modification is 64% compared to a biphasic microfiber, where both phases are modified with the laminin peptide.

Microfibers that are placed in close proximity to modified phases of other microfibers showed a higher number of non-specifically attached cells than individual fibers placed in isolation, possibly due to multiple interactions between the cells and the ligands on the neighboring fibers. In contrast, microfiber scaffolds with no acetylene-PLGA in either phase, but subjected to copper-catalyzed Huisgen heterocycloaddition chemistry with the peptide, show negligible cell adhesion on the fiber surface (FIG. 9F). Similarly, microfibers with acetylene-PLGA in both phases, but without peptide modification, do not appear to support cell adhesion. These control data confirm that cell adhesion indeed appears to be caused by specific interactions between the cell receptors and the laminin peptide.

Thus, a novel fabrication process for cell culture scaffolds is provided by the present teachings that enable superior control of physical, chemical, and biological properties. The resulting multiphasic microfiber scaffolds provide new options for overcoming key challenges in a number of biotechnological applications, namely providing spatial control over cell adhesive properties of three-dimensional scaffolds.

Another application for the multiphasic microfibers is for cell cultures, such as stem cell cultures. For example, a multiphasic fiber can be provided having different phases composed of materials having independent and distinct degradation kinetics. Different molecular signals that direct stem cell differentiation are optionally encapsulated in each phase. Thus, such scaffolds or cellular support structures enable stem cell differentiation into a given cell line while providing a three dimensional environment for them to grow and proliferate. Other examples of multiphasic microfibers are provided herein.

### Example 8

### Dual Protein Delivery Microfibers

A solution of 5-15 wt. % polyethylene oxide (PEO) (100 kD) and bone morphogenetic protein (BMP-2) in water is co-jetted with a solution of 5-15 wt. % PEO (100 kD) and transcription factor SRY-related HMG-box gene 9 (Sox-9). The protein content can vary between about 0.01 to about 25% relative to PEO. Two parallel polymer flows are introduced in a nozzle that contains to inlets in a side-by-side geometry, as described above in Example 1. A polymer thread is ejected from the droplet resulting in biphasic fibers with one phase having BMP-2 as an active ingredient and the other phase including Sox-9 as an active ingredient. The controlled delivery of the proteins facilitates specific tissue formation: BMP-2 (bone) and Sox-9 (cartilage).

### Example 9

### Magnetic Microfibers for MRI Imaging

A 10 wt % aqueous solution of 95 wt. % PAA and 5 wt. % PAAm-co-AA is co-jetted with an aqueous solution of PAAm-coAA which comprises an active ingredient including magnetite nanocrystals homogeneously suspended in the polymer solution. The content of the magnetite nanocrystals can vary from about 0.05 to about 25 wt. % relative to PAAm-co-AA. Two parallel polymer flows are introduced in a nozzle that contains to inlets in a side-by-side geometry, as described above in Example 1. A biphasic nano-component with magnetite nanocrystals as the active ingredient in one phase is formed. These multiphasic microfibers show a clear response to application of a magnetic field. Thus, such multiphasic microfibers can be employed in conjunction with magnetic resonance imaging (MRI) for medical diagnosis and surgical placement and mapping applications.

### Example 10

### Protein-Containing Multiphasic Microfibers

A 10 wt % aqueous solution of 95 wt. % PAA and 5 wt. % PAAm-co-AA is co-jetted with an aqueous solution of PAAm-coAA which also contains PEO at about 0.05 to about 75 wt % relative to PAAm-co-AA. The PEO contains vascular endothelial growth factor (VEGF) as an active ingredient, at a concentration of about 0.1 to about 20 wt. % relative to PEO. A side-by-side jetting apparatus is used, as described above in Example 1. Biphasic microfibers having PEO and VEGF protein in one phase are created. In this manner, the VEGF biological function is preserved during the formation process and after storage for several weeks.

### Example 11

### Hydrophobic/Hydrophilic Multiphasic Microfibers

A solution of 5 wt. % PLGA in chloroform is co-jetted with a solution of 15-18 wt. % PLGA in chloroform and a polylactic acid (PLA) having (on average) at least one acetylene group per molecule. A side-by-side jetting apparatus is used, as described above in Example 1. Biphasic microfibers are formed, where one phase has PLGA (50:50) and the other phase has PLGA (85:15) with acetylene-modified PLA. A functionalized surface is provided for one phase by subsequent reaction/conversion of acetylene with an azide-polyoxyethylene glycol (Azide-PEG) ligand, which results in PEG-ylation of a surface (a hemisphere) of the biphasic microfibers, providing the functionalized side with hydrophilic properties and the PLGA phase with hydrophobic properties.

### Example 12

### Hydrophobic/Hydrophilic Multiphasic Microfibers

A solution of 5-10 wt. % polydimethylsiloxane (PMDS) in chloroform is co-jetted with an aqueous solution of 5 wt. % of collagen containing basic fibroblast growth factor (BFGF) as the active biofunctional ingredient. The forming apparatus is the same as that discussed above for Example 1. Biphasic microfibers are formed where one phase comprises hydrophobic PDMS and the other phase comprises collagen/BFGF, which could be used in various tissue applications. Further, such multiphasic microfibers can be used in various applications, such as for sprayable wound coverage.

### Example 13

### Hydrophobic/Hydrophilic Multiphasic Microfibers

A solution of 5-10 wt. % polydimethylsiloxane (PMDS) in chloroform is co-jetted with an aqueous solution of 5 wt. % of collagen containing an active ingredient comprising genetically-modified adenovirus. The forming apparatus is the same as that discussed above for Example 1. Biphasic microfibers are formed with one phase comprising PDMS (hydrophobic) and the other phase having collagen and the adenovirus. Such multiphasic microfibers can be used for transfection of cells in the context of gene therapy, for example, in implants.

### Example 14

### Fibroblast Adhesion on Multiphasic Microfibers

Glass slides are modified with poly(ethylene glycol) methacrylate (PEGMA) using an atom transfer radical polymerization (ATRP) technique before collecting PLGA microfibers. Two different samples are prepared: a first sample of biphasic fibers formed with PLGA in both phases per the apparatus of Example 1, and a second sample having biphasic fibers with a first phase comprising PLGA and the neighboring phase modified with polyethylene glycol (PEG). The fibers are prepared from the following solutions: one phase contains 18:100 w/w polymer:solvent with the polymer being PLGA 85:15 with the solvent being 95:5 Chloroform:DMF by volume. The other phase contains 18:100 w/w ratio of polymer:solvent, with the polymer being a 30:100 w/w mixture of poly(lactide-co-propargyl glycolide):PLGA85:15 and the solvent, 95:5 Chloroform:DMF by volume.

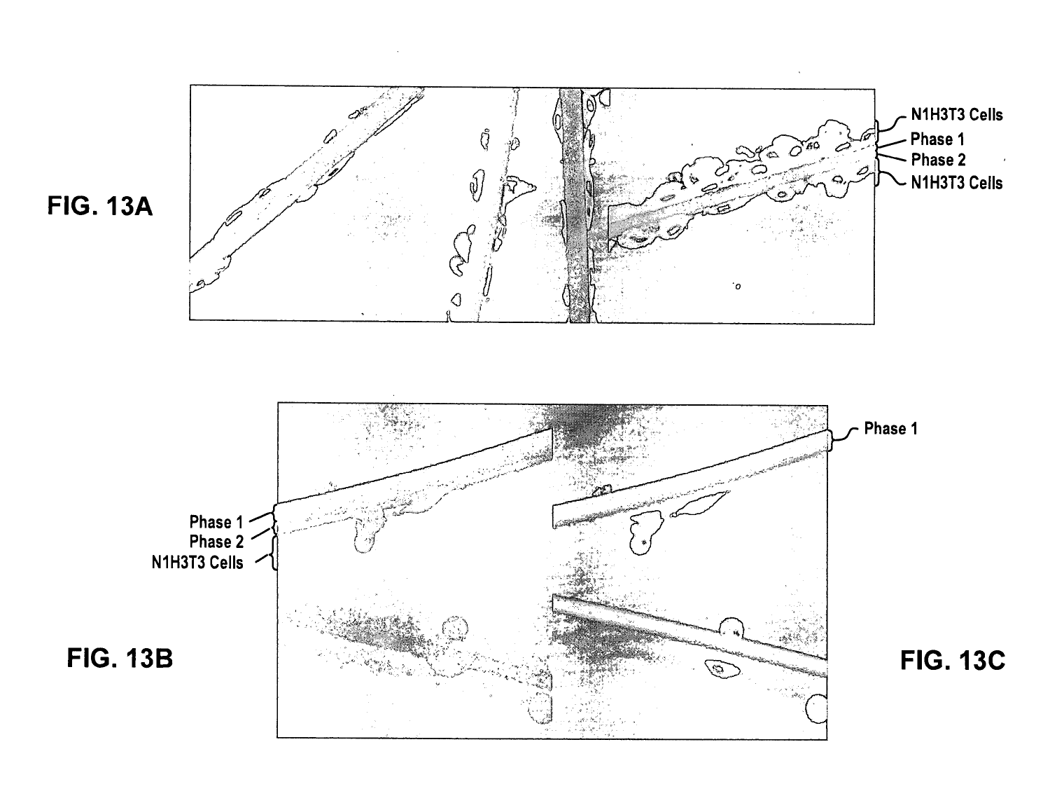

The microfibers are placed in a 12-well plate and fibronectin (from human plasma, commercially available from Invitrogen) is added to the samples at a concentration of 50 μg/mL in phosphate-buffered saline (PBS) for 1 hour. The samples are then washed with 0.02% Tween-20/0.1% BSA/PBS solution for 3 times with 5 minutes interval. A NIH 3T3 fibroblast suspension in serum-free media is added to the samples at a concentration of 1.0×10⁵ cells/cm² under 37° C./5% CO₂ and incubated for 6 hours under culture conditions. A live-cell actin stain BODIPY-TMR-Cytochalasin D (commercially available from Invitrogen) is added to the media for 20 minutes under culture conditions before imaging. FIG. 13A shows the first sample demonstrating that the NIH 3T3 fibroblasts adhere to both phases (Phases 1 and 2) of the PLGA microfibers. FIGS. 13 B-C show the second sample demonstrating selective adhesion of the NIH 3T3 fibroblasts on the PLGA phase (Phase 2) of the microfiber (FIG. 13B), while FIG. 13C is oriented to show only the surface of the PEG-PLGA phase (Phase 1) surface (so that PLGA—Phase 2 is hidden), which has no fibroblast growth on the PEG-PLGA surface.

In terms of a cell substrate or tissue scaffold design, a series of new design parameters are introduced that provide the next generation of scaffolds for tissue engineering, namely (i) internal fiber architecture, (ii) spatial configuration of the individual fiber phases, (iii) long-range alignment of individual phases within a scaffold and (iv) the precise control of the relative size of individual phases. The ability to precisely control the internal architecture of multiphasic fiber scaffolds gives rise to a number of secondary control parameters, such as controlled chemical composition, optical anisotropy, or spatially controlled surface modification, which provide multiphasic microfiber scaffolds for guided cell adhesion.

Such multiphasic microfiber cellular support structures or scaffolds are important not only for fundamental biological studies, but also in a series of biotechnological applications including high-throughput screening, co-culture of multiple cell types, or regenerative medicine, by way of non-limiting example. Thus, electrohydrodynamic jetting processes of the present disclosure can be used with a wide range of specialty and non-specialty materials including many currently Federal Drug Administration (FDA) approved polymers. Each respective phase can be designed independently from the other phase(s) enabling the combination of multiple material functions during design. The present disclosure provides for a high degree of control over shape, size, and/or orientation of phases during the formation of biodegradable multiphasic nanofibers and microfibers. In certain aspects, biphasic electrified jetting process provides fabrication of multiphasic micro-particles of different shapes or sizes. Selective modification of each phase with ligands or biofunctional substances provides the ability to spatially control and guide cell growth, regeneration, differentiation, proliferation, and/or repair, for example. Thus, novel scaffolds or cellular support substrates are formed that enable spatially controlled cell proliferation and growth from multiphasic microfibers, which are of significant value to regenerative medicine, among others.

The foregoing description of the embodiments has been provided for purposes of illustration and description. It is not intended to be exhaustive or to limit the present teachings. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the present teachings, and all such modifications are intended to be included within the scope of the disclosure.

What is claimed is:

1. A multiphasic microfiber defining a longitudinal major axis and comprising at least one biocompatible material, a first phase, and at least one additional phase distinct from said first phase, wherein at least a portion of said first phase and at least a portion of said additional phase form exposed surfaces to a surrounding environment, wherein the multiphasic microfiber supports and/or promotes cell growth, cell proliferation, cell differentiation, cell repair, and/or cell regeneration.

2. The multiphasic microfiber of claim 1, wherein said first phase and said at least one additional phase are substantially aligned from a first end to a second end of the microfiber along said longitudinal major axis.

3. The multiphasic microfiber of claim 1, wherein at least one of said first phase and said at least one additional phase further comprises a biofunctional agent.

4. The multiphasic microfiber of claim 3, wherein said biofunctional agent is selected from the group consisting of: growth factors, growth factor receptors, transcriptional activators, translational promoters, anti-proliferative agents, growth hormones, anti-rejection drugs, anti-thrombotic agents, anti-coagulants, stem cell or gene therapy agents, antioxidants, free radical scavengers, nutrients, co-enzymes, ligands, cell adhesion peptides, peptides, proteins, nucleic acids, DNA, RNA, polysaccharides, sugars, nutrients, hormones, antibodies, immunomodulating agents, growth factor inhibitors, growth factor receptor antagonists, transcriptional repressors, translational repressors, replication inhibitors, inhibitory antibodies, cytotoxin, hormonal agonists, hormonal antagonists, inhibitors of hormone biosynthesis and processing, antigestagens, antiandrogens, anti-inflammatory agents, non-steroidal anti-inflammatory agents (NSAIDs), COX-I and II inhibitors, antimicrobial agents, antiviral agents, antifungal agents, antibiotics, antineoplastic/antiproliferative/anti-miotic agents, anesthetic, analgesic or pain-killing agents, antipyretic agents, prostaglandin inhibitors, platelet inhibitors, DNA de-methylating agents, cholesterol-lowering agents, vasodilating agents, endogenous vasoactive interference agents, angiogenic substances, cardiac failure active ingredients, targeting toxin agents; aptamers, quantum dots, nano-materials, nano-crystals, and combinations thereof.

5. The multiphasic microfiber of claim 3, wherein said biofunctional agent is selected from the group consisting of: erythropoietin, stem cell factor (SCF) vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factor (CGF), stem cell factor (SCF), platelet-derived growth factor (PDGF), endothelial cell growth supplement (EGGS), colony stimulating factor (CSF), growth differentiation factor (GDF), integrin modulating factor (IMF), calmodulin (CaM), thymidine kinase (TK), tumor necrosis factor (TNF), growth hormone (GH), bone morphogenic proteins (BMP), matrix metalloproteinase (MMP), tissue inhibitor matrix metalloproteinase (TIMP), interferon, interleukins, cytokines, collagen, elastin, fibrillins, nectins, fibronectin, hemonectin, laminin, glycosaminoglycans, thrombospondin, heparan sulfate, dermantan, chondrotin sulfate (CS), hyaluronic acid (HA), vitronectin, proteoglycans, transferrin, cytotactin, tenascin, lymphokines, YIGSR cell adhesion peptide, IKVAV cell adhesion peptide, RGD cell adhesion peptide, RAD cell adhesion peptide, RGDS cell adhesion peptide, RGES cell adhesion peptide, EILDV cell adhesion peptide, EILEVPST cell adhesion peptide, CS-1 fragment cell adhesion peptide, REDV cell adhesion peptide, CS-5 fragment cell adhesion peptide, neural cell adhesion molecules (N-CAMS), intercellular cell adhesion molecules (ICAMS), integrins, selectins, cadherins, vascular cell adhesion molecule (VCAM), platelet-endothelial cell adhesion molecule (PECAM), and combinations thereof.

6. The multiphasic microfiber of claim 3 wherein said biofunctional agent is a first biofunctional agent and the microfiber further comprises a second distinct biofunctional agent.

7. The multiphasic microfiber of claim 3 wherein said biofunctional agent is a moiety disposed on a surface of said first phase selected from the group: proteins, peptides, polysaccharides, sugars, toxins, antibodies, aptamers, and combinations thereof.

8. The multiphasic microfiber of claim 1, wherein said at least one biocompatible material comprises a polyester polymer selected from the group consisting of polylactides, polyglycolides, co-polymers, derivatives, and combinations thereof.

9. The multiphasic microfiber of claim 1, wherein said at least one biocompatible material comprises a polymer selected from the group consisting of polylactic acid, polycaprolactone, polyglycolic acid, poly(lactide-co-glycolide polymer (PLGA), and copolymers, derivatives, and combinations thereof.

10. The multiphasic microfiber of claim 1, wherein at least one of said exposed surfaces is treated after formation of the microfiber to modify the chemical or physical characteristics of said surface.

11. A three-dimensional cellular scaffold structure comprising at least two multiphasic microfibers wherein each of said two multiphasic microfibers defines a longitudinal major axis and comprises a first phase and at least one additional phase distinct from said first phase, wherein at least a portion of said first phase and said at least one additional phase has an exposed surface to an external surrounding environment and comprises a biocompatible material, so that the cellular scaffold structure supports and/or promotes cell growth, cell proliferation, cell differentiation, cell repair, and/or cell regeneration in three-dimensions.

12. The three-dimensional cellular scaffold structure of claim 11, wherein said cell is selected from the group consisting of: autologous cells, allogeneic cells, xenogeneic cells, genetically engineered cells, progenitor cells, mesenchymal stem cells, hematopoietic stem cells, neuronal stem cells, stromal cells, parenchymal cells, undifferentiated cells, fibroblasts, macrophage cells, satellite cells, nerve cells, hepatocytes, epithelial cells, endothelial cells, immune system cells, and combinations thereof.

13. The three-dimensional cellular scaffold structure of claim 11, wherein a first microfiber of the at least two multiphasic microfibers defines a first longitudinal major axis and a second microfiber of the at least two multiphasic microfibers defines a second longitudinal major axis, wherein said first and second longitudinal major axes are substantially aligned with one another.

14. A medical implant comprising the three-dimensional cellular scaffold structure of claim 11 suitable for implantation into a human.

15. A cell culture device comprising the three-dimensional cellular scaffold structure of claim 11.

16. The three-dimensional cellular scaffold structure of claim 11, further comprising a biofunctional agent selected from the group consisting of: growth factors, growth factor receptors, transcriptional activators, translational promoters, anti-proliferative agents, growth hormones, anti-rejection drugs, anti-thrombotic agents, anti-coagulants, stem cell or gene therapy agents, antioxidants, free radical scavengers, nutrients, co-enzymes, ligands, cell adhesion peptides, peptides, proteins, nucleic acids, DNA, RNA, polysaccharides, sugars, nutrients, hormones, antibodies, immunomodulating agents, growth factor inhibitors, growth factor receptor antagonists, transcriptional repressors, translational repressors, replication inhibitors, inhibitory antibodies, cytotoxin, hormonal agonists, hormonal antagonists, inhibitors of hormone biosynthesis and processing, antigestagens, antiandrogens, anti-inflammatory agents, non-steroidal anti-inflammatory agents (NSAIDs), COX-I and II inhibitors, antimicrobial agents, antiviral agents, antifungal agents, antibiotics, antineoplastic/antiproliferative/anti-miotic agents, anesthetic, analgesic or pain-killing agents, antipyretic agents, prostaglandin inhibitors, platelet inhibitors, DNA de-methylating agents, cholesterol-lowering agents, vasodilating agents, endogenous vasoactive interference agents, angiogenic substances, cardiac failure active ingredients, targeting toxin agents; aptamers, quantum dots, nano-materials, nano-crystals, and combinations thereof.

17. The three-dimensional cellular scaffold structure of claim 11, further comprising a biofunctional agent selected from the group consisting of: erythropoietin, stem cell factor (SCF) vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factor (CGF), stem cell factor (SCF), platelet-derived growth factor (PDGF), endothelial cell growth supplement (EGGS), colony stimulating factor (CSF), growth differentiation factor (GDF), integrin modulating factor (IMF), calmodulin (CaM), thymidine kinase (TK), tumor necrosis factor (TNF), growth hormone (GH), bone morphogenic proteins (BMP), matrix metalloproteinase (MMP), tissue inhibitor matrix metalloproteinase (TIMP), interferon, interleukins, cytokines, collagen, elastin, fibrillins, nectins, fibronectin, hemonectin, laminin, glycosaminoglycans, thrombospondin, heparan sulfate, dermantan, chondrotin sulfate (CS), hyaluronic acid (HA), vitronectin, proteoglycans, transferrin, cytotactin, tenascin, lymphokines, YIGSR cell adhesion peptide, IKVAV cell adhesion peptide, RGD cell adhesion peptide, RAD cell adhesion peptide, RGDS cell adhesion peptide, RGES cell adhesion peptide, EILDV cell adhesion peptide, EILEVPST cell adhesion peptide, CS-1 fragment cell adhesion peptide, REDV cell adhesion peptide, CS-5 fragment cell adhesion peptide, neural cell adhesion molecules (N-CAMS), intercellular cell adhesion molecules (ICAMS), integrins, selectins, cadherins, vascular cell adhesion molecule (VCAM), platelet-endothelial cell adhesion molecule (PECAM), and combinations thereof.

18. The three-dimensional cellular scaffold structure of claim 11, wherein said biofunctional agent is a moiety disposed on a surface of said first phase and is selected from the group: proteins, peptides, polysaccharides, sugars, toxins, antibodies, aptamers, and combinations thereof.

19. A method for treating a defective, diseased, damaged or ischemic tissue or organ in a mammal comprising implanting the three-dimensional cellular scaffold structure of claim 11 into the mammal.

20. A method of making a multiphasic microfiber for a tissue scaffold and/or cellular support structure, the method comprising:
forming a plurality of multiphasic microfibers by jetting two or more liquid streams together and passing them through an electric field generated by electrodes sufficient to form a cone jet that forms said plurality of multiphasic microfibers, each respectively having a first phase and at least one additional phase distinct from said first phase, each forming exposed surfaces of the microfiber, wherein each multiphasic microfiber of the plurality comprises a biocompatible material for supporting and/or promoting cell growth, cell proliferation, cell differentiation, cell repair, and/or cell regeneration.

21. The method of claim 20, wherein a first microfiber of the plurality defines a first longitudinal major axis and a second microfiber of the plurality defines a second longitudinal major axis, wherein said first and second longitudinal major axes are substantially aligned with one another after said forming.

22. The method of claim 20, wherein said biocompatible materials of said first phase and said at least one additional phase respectively comprise a poly(lactic co-glycolide) polymer present in said liquid streams at greater than about 4% by weight of the total jetting liquid stream, wherein said jetting occurs at a flow rate of greater than or equal to about 0.7 milliliters per hour.

23. The method of claim 20, further comprising a biofunctional agent selected from the group consisting of: growth factors, growth factor receptors, transcriptional activators, translational promoters, anti-proliferative agents, growth hormones, anti-rejection drugs, anti-thrombotic agents, anti-coagulants, stem cell or gene therapy agents, antioxidants, free radical scavengers, nutrients, co-enzymes, ligands, cell adhesion peptides, peptides, proteins, nucleic acids, DNA, RNA, polysaccharides, sugars, nutrients, hormones, antibodies, immunomodulating agents, growth factor inhibitors, growth factor receptor antagonists, transcriptional repressors, translational repressors, replication inhibitors, inhibitory antibodies, cytotoxin, hormonal agonists, hormonal antagonists, inhibitors of hormone biosynthesis and processing, antigestagens, antiandrogens, anti-inflammatory agents, non-steroidal anti-inflammatory agents (NSAIDs), COX-I and II inhibitors, antimicrobial agents, antiviral agents, antifungal agents, antibiotics, antineoplastic/antiproliferative/anti-miotic agents, anesthetic, analgesic or pain-killing agents, antipyretic agents, prostaglandin inhibitors, platelet inhibitors, DNA de-methylating agents, cholesterol-lowering agents, vasodilating agents, endogenous vasoactive interference agents, angiogenic substances, cardiac failure active ingredients, targeting toxin agents; aptamers, quantum dots, nano-materials, nano-crystals, and combinations thereof.
